(19) United States
(12) Patent Application Publication
(10) Pub. No.: US 2011/0280831 A1
(43) Pub. Date: Nov. 17, 2011

(54) COMBINATIONS COMPRISING METHOTREXATE AND DHODH INHIBITORS

(76) Inventors: Nuria Godessart Marina, Barcelona (ES); Maria Pilar Pizcueta Lalanza, Barcelona (ES)

(21) Appl. No.: 13/145,628

(22) PCT Filed: Jan. 19, 2010
(86) PCT No.: PCT/EP2010/000270

§ 371 Date: Jul. 21, 2011

(30) Foreign Application Priority Data
Jan. 21, 2009 (EP) 09382006.6

(51) Int. Cl. A61K 31/519; A61K 31/55; A61K 39/395; A61K 38/21; A61P 1/16; A61P 31/14; A61P 35/00; A61P 29/00; A61P 17/06; A61P 19/00; A61P 25/00; A61P 37/00; A61P 19/04; A61P 17/00; A61K 31/5377

(52) U.S. Cl. 424/85.6; 514/249; 514/235.5; 514/217.04; 424/145.1; 424/144.1

(57) ABSTRACT

The present invention provides a combination which comprises (a) methotrexate and (b) a non-hepatotoxic DHODH inhibitor of formula (I): wherein: R¹ is selected from the group consisting of hydrogen atoms, halogen atoms, C₁₋₄ alkyl, C₃₋₄ cycloalkyl, —CF₃ and —OCF₃, R² is selected from the group consisting of hydrogen atoms, halogen atoms and C₁₋₄ alkyl groups, R³ is selected from the group consisting of —COOR⁵, —CONHR⁵, tetrazolyl, —SO₂NHR⁵ and —CONHSO₂R⁵ groups, wherein R⁵ is selected from the group consisting of a hydrogen atom and linear or branched C₁₋₄ alkyl groups, R⁴ is selected from the group consisting of a hydrogen atom and a C₁₋₄ alkyl group, R⁹ is selected from the group consisting of a hydrogen atom and a phenyl group, G¹ represents a group selected from N and CR⁶ wherein R⁶ is selected from the group consisting of hydrogen atoms, halogen atoms, C₁₋₄ alkyl, C₃₋₄ cycloalkyl, C₁₋₄ alkoxy, —CF₃, —OCF₃, monocyclic N-containing C₅₋₇ heteroaryl, monocyclic N— containing C₃₋₇ heterocyclyl groups and C₆₋₁₀ aryl groups which C₆₋₁₀ aryl groups are optionally substituted with one or more substituents selected from halogen atoms and C₁₋₄ alkyl groups, G¹ represents a group selected from N and CR⁶ wherein R⁶ is selected from the group consisting of hydrogen atoms, halogen atoms, C₁₋₄ alkyl, C₃₋₄ cycloalkyl, C₁₋₄ alkoxy, —CF₃, —OCF₃, mono-cyclic N-containing C₅₋₇ heteroaryl, monocyclic N— containing C₃₋₇ heterocyclyl groups and C₆₋₁₀ aryl groups which C₆₋₁₀ aryl groups are optionally substituted with one or more substituents selected from halogen atoms and C₁₋₄ alkyl groups, G² represents a group selected from: a hydrogen atom, a hydroxy group, a halogen atom, a C₃₋₄ cycloalkyl group, a C₁₋₄ alkoxy group and —NR^{a}R^{b}, wherein R^{a} represents a C₁₋₄ alkyl group and R^{b} is selected from a group consisting of C₁₋₄ alkyl group and C₁₋₄alkoxy-C₁₋₄ alkyl group, or Ra and Rb together with the nitrogen atom to which they are attached form a saturated 6 to 8 membered heterocyclic ring optionally containing one oxygen atom as an additional heteroatom, a monocyclic or bicyclic 5 to 10 membered heteroaromatic ring containing one or more nitrogen atoms which is optionally substituted by one or more substituents selected from halogen atoms, C₁₋₄ alkyl, C₁₋₄ alkoxy, C₃₋₄ cycloalkyl, C₃₋₄ cycloalkoxy, —CF₃, —OCF₃, and —CONR⁷R⁸, wherein R⁷ and R⁸ are independently selected from hydrogen atom, linear or branched C₁₋₄ alkyl groups, C₃₋₇ cycloalkyl groups, or R⁷ and R⁸ together with the nitrogen atom to which they are attached form a group of formula wherein n is an integer from 0 to 3, and a phenyl group which is optionally substituted by one or more substituents selected from halogen atoms, C₁₋₄ alkyl, hydroxyl, C₁₋₄ alkoxy, C₃₋₄ cycloalkyl, C₃₋₄ cycloalkoxy, cyano, —CF₃, —OCF₃, —CONR⁷R⁸, oxadiazolyl, triazolyl, pyrazolyl and imidazolyl groups, which oxadiazolyl, triazolyl, pyrazolyl and imidazolyl groups are optionally substituted by C₁₋₄ alkyl or C₃₋₇ cycloalkyl groups and wherein R⁷ and R⁸ are independently selected from hydrogen atom, linear or branched C₁₋₄ alkyl groups, C₃₋₇ cycloalkyl groups, or R⁷ and R⁸ together with the nitrogen atom to which they are attached form a group of formula wherein n is an integer from 0 to 3 or, when G′ represents CR⁶, G² together with R⁶ forms a non-aromatic C₅₋₁₀ carbocyclic group or a C₆₋₁₀ aryl group, and the pharmaceutically acceptable salts and N-oxides thereof.

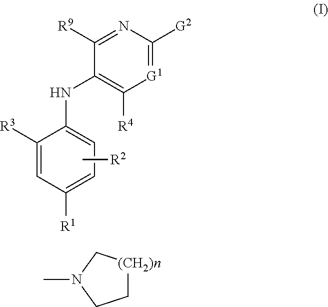

The present invention relates to new combinations of methotrexate with DHODH inhibitors. These combinations are useful in the treatment, prevention or suppression of diseases and disorders known to be susceptible to improvement with methotrexate and/or by inhibition of dihydroorotate dehydrogenase, such as autoimmune diseases, immune and inflammatory diseases, destructive bone disorders, malignant neoplastic diseases, angiogenic-related disorders, viral diseases, and infectious diseases.

### BACKGROUND OF THE INVENTION

Methotrexate (MTX) is an antimetabolite and immunomodulator that affects many intracellular pathways of purine metabolism. It is effective in reducing the signs and symptoms of rheumatoid arthritis (RA), as well as in slowing or halting radiographic damage. Due to its efficacy, ease of administration and relatively low cost, MTX has become the first-line oral therapy in most patients with RA. In those patients who have an incomplete response to MTX, another DMARD (disease modifying anti-rheumatic drug) is added on top of it. Thus, combination therapy with MTX is more and more frequent in the clinical practice.

Leflunomide is an example of such a DMARD. It was approved in September 1998 for use in RA. It has been shown to reduce the signs and symptoms of the disease, to inhibit structural damage (evidenced by X-ray erosions and joint space narrowing) and to improve physical function. Teriflunomide is the active metabolite of Leflunomide.

Methotrexate is thought to act primarily on purine pathways of cellular metabolism, whereas Leflunomide affects pyrimidine pathways. Given the diverse intracellular pathways affected by both drugs, the combination of Leflunomide and methotrexate has the potential for biochemical synergy. In fact, it has been reported that the combination of both agents led to considerable clinical improvement (see for example, Weinblatt M E et al. “Pharmacokinetics, safety, and efficacy of combination treatment with methotrexate and leflunomide in patients with active rheumatoid arthritis”. Arthritis Rheum 1999; 42 (7): 1322-8 and Kremer J M et al. “Concomitant Leflunomide therapy in patients with active rheumatoid arthritis despite stable doses of methotrexate”. Ann. Intern. Med., 2002; 137, 726-733).

Unfortunately, both methotrexate and leflunomide have serious adverse effects, in particular hepatotoxicity. Methotrexate may cause fatal liver damage such as fibrosis and cirrhosis after prolonged use. Liver enzyme increases are frequently seen during treatment with methotrexate. Hence, regular and careful monitoring of patients taking MTX is essential, particularly when MTX is combined with other DMARDs.

The most common reported adverse events of Leflunomide include diarrhoea, dyspepsia, rash, hair loss, hypertension and elevated hepatic enzymes. The hepatotoxicity potential is of special relevance and regular laboratory tests, including blood tests of liver function, must be performed for all patients taking this medication. Leflunomide is not recommended for use in patients with evidence of hepatitis B or C infection or significant hepatic impairment.

Clinical trials have reported that the number of patients experiencing an increase in liver markers (measured as transaminase levels) is notably higher in the group of Leflunomide plus MTX than in the group of MTX alone. The product information for Leflunomide warns against combination with methotrexate on the basis that such combination therapy can lead to additive or even synergistic hepatotoxicity.

The mechanism responsible for the hepatotoxicity of leflunomide, and in particular of its active metabolite, teriflunomide, is unknown, but it has been attributed to its activity as inhibitor of dihydroorotate dehydrogenase (DHODH). Liver toxicity has thus been identified as an adverse effect directly derived from the mechanism of action of DHODH-inhibitors, which has hampered the development of this class of compounds.

### DESCRIPTION OF THE INVENTION

It has now been found that, contrary to general belief, inhibition of DHODH is not responsible for the liver damage produced by leflunomide and that certain non-hepatotoxic DHODH inhibitors are particularly suitable for combination with methotrexate.

It is known that inhibition of DHODH produces immunosuppressant and antiproliferative effects. DHODH inhibitors can therefore be used as immunosuppressants and as antiproliferatives in the treatment of autoimmune, inflammatory and proliferative diseases, like RA.

The present invention is based on the surprising finding that the inhibition of DHODH is not linked to hepatotoxicity and, consequently, DHODH inhibitors devoid of hepatotoxic potential represent an important contribution to the treatment of these diseases, due to their advantageous combinability with MTX, the most commonly used first-line drug in RA treatment.

We have developed an in vivo model of hepatotoxicity assessment in mice, in which test compounds are administered by intraperitoneal route to maximise liver exposure. In this model, Teriflunomide, the active metabolite of Leflunomide, has shown a drastic increase in the levels of transaminases and bilirrubin in plasma, whereas DHODH inhibitors do not show an increase in any of the plasma liver markers in the same model, while maintaining their efficacy in arthritis.

Thus, the present invention is directed to a combination which comprises (a) methotrexate and (b) a non-hepatotoxic DHODH inhibitor of formula (I):

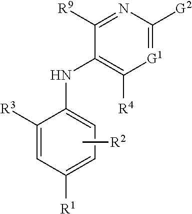

wherein:
R¹ is selected from the group consisting of hydrogen atoms, halogen atoms, C₁₋₄ alkyl, C₃₋₄ cycloalkyl, —CF₃ and —OCF₃,
R² is selected from the group consisting of hydrogen atoms, halogen atoms and C₁₋₄ alkyl groups,
R³ is selected from the group consisting of —COOR⁵, —CONHR⁵, tetrazolyl,
—SO₂NHR⁵ and —CONHSO₂R⁵ groups, wherein R⁵ is selected from the group consisting of a hydrogen atom and linear or branched C₁₋₄ alkyl groups,
R⁴ is selected from the group consisting of a hydrogen atom and a C₁₋₄ alkyl group,
R⁹ is selected from the group consisting of a hydrogen atom and a phenyl group,
G¹ represents a group selected from N and CR⁶ wherein R⁶ is selected from the group consisting of hydrogen atoms, halogen atoms, C₁₋₄ alkyl, C₃₋₄ cycloalkyl, C₁₋₄ alkoxy, —CF₃, —OCF₃, monocyclic N-containing C₆₋₇ heteroaryl, monocyclic N-containing C₃₋₇ heterocyclyl groups and C₆₋₁₀ aryl groups which C₆₋₁₀ aryl groups are optionally substituted with one or more substituents selected from halogen atoms and C₁₋₄ alkyl groups,
G² represents a group selected from:
   a hydrogen atom, a hydroxy group, a halogen atom, a C₃₋₄ cycloalkyl group, a C₁₋₄ alkoxy group and —NR^{a}R^{b}, wherein
      R^{a} represents a C₁₋₄ alkyl group and R^{b} is selected from a group consisting of C₁₋₄ alkyl group and C₁₋₄ alkoxy-C₁₋₄ alkyl group, or
      R^{a} and R^{b} together with the nitrogen atom to which they are attached form a saturated 6 to 8 membered heterocyclic ring optionally containing one oxygen atom as an additional heteroatom,
   a monocyclic or bicyclic 5 to 10 membered heteroaromatic ring containing one or more nitrogen atoms which is optionally substituted by one or more substituents selected from halogen atoms, C₁₋₄ alkyl, C₁₋₄ alkoxy, C₃₋₄ cycloalkyl, C₃₋₄ cycloalkoxy, —CF₃, —OCF₃, and —CONR⁷R⁸, wherein R⁷ and R⁸ are independently selected from hydrogen atom, linear or branched C₁₋₄ alkyl groups, C₃₋₇ cycloalkyl groups, or R⁷ and R⁸ together with the nitrogen atom to which they are attached form a group of formula

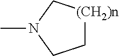

wherein n is an integer from 0 to 3,
and
   a phenyl group which is optionally substituted by one or more substituents selected from halogen atoms, C₁₋₄ alkyl, hydroxyl, C₁₋₄ alkoxy, C₃₋₄cycloalkyl, C₃₋₄ cycloalkoxy, cyano, —CF₃, —OCF₃, —CONR⁷R⁸, oxadiazolyl, triazolyl, pyrazolyl and imidazolyl groups, which oxadiazolyl, triazolyl, pyrazolyl and imidazolyl groups are optionally substituted by C₁₋₄ alkyl or C₃₋₇ cycloalkyl groups and wherein R⁷ and R⁸ are independently selected from hydrogen atom, linear or branched C₁₋₄ alkyl groups, C₃₋₇ cycloalkyl groups, or R⁷ and R⁸ together with the nitrogen atom to which they are attached form a group of formula

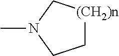

wherein n is an integer from 0 to 3
or, when G′ represents CR⁶, G² together with R⁶ forms a non-aromatic C₅₋₁₀ carbocyclic group or a C₆₋₁₀aryl group,
   and the pharmaceutically acceptable salts and N-oxides thereof.

As used herein the term alkyl embraces optionally substituted, linear or branched hydrocarbon radicals having 1 to 4 carbon atoms. Preferred substituents on the alkyl groups are halogen atoms and hydroxy groups.

Examples include methyl, ethyl, n-propyl, i-propyl, n-butyl, sec-butyl and tert-butyl radicals.

As used herein the term alkoxy embraces optionally substituted, linear or branched oxygen containing radicals each having 1 to 4 carbon atoms. Preferred substituents on the alkoxy groups are halogen atoms and hydroxy groups.

Examples include methoxy, ethoxy, n-propoxy, i-propoxy, n-butoxy, sec-butoxy and tert-butoxy radicals.

As used herein, the term cycloalkyl embraces optionally substituted saturated carbocyclic radicals and, unless otherwise specified, a cycloalkyl radical typically has from 3 to 7 carbon atoms, preferably from 3 to 4 carbon atoms.

Examples include cyclopropyl, cyclobutyl, cyclopentyl, cyclohexyl and cycloheptyl. When a cycloalkyl radical carries 2 or more substituents, the substituents may be the same or different. Preferred substiuents on the cycloalkyl groups are halogen atoms and hydroxy groups.

As used herein, the term cycloalkoxy embraces saturated oxy-containing carbocyclic radicals and, unless otherwise specified, a cycloalkoxy radical typically has from 3 to 8 carbon atoms, preferably from 3 to 4 carbon atoms.

Examples include cyclopropyloxy, cyclobutyloxy, cyclopentyloxy, cyclohexyloxy and cycloheptyloxy. When a cycloalkoxy radical carries 2 or more substituents, the substituents may be the same or different. Preferred substiuents on the cycloalkoxy groups are halogen atoms and hydroxy groups.

As used herein, the term aryl radical embraces typically optionally substituent C₆-C₁₀ monocyclic or polycyclic aryl radical such as phenyl, naphthyl, anthranyl and phenanthryl. Phenyl is preferred.

A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁-C₄ alkyl groups, C₁-C₄ alkoxy groups and C₁-C₄ hydroxyalkyl groups. When an aryl radical carries 2 or more substituents, the substituents may be the same or different. Unless otherwise specified, the substituents on an aryl group are typically themselves unsubstituted.

As used herein, the terms heteroaryl and heteroaromatic ring are used interchangeably and are typically 5- to 14-membered ring systems, preferably 5- to 10-membered ring systems, comprising at least one heteroaromatic ring and containing at least one heteroatom selected from O, S and N. A heteroaryl radical may be a single ring (monocyclic) or two or more fused rings (polycyclic) wherein at least one ring contains a heteroatom.

As used herein, the term heterocyclyl radical is typically a non-aromatic, saturated or unsaturated C₃-C₁₀ carbocyclic ring system, such as a 5, 6 or 7 membered radical, in which one or more, for example 1, 2, 3 or 4 of the carbon atoms preferably 1 or 2 of the carbon atoms are replaced by a heteroatom selected from N, O and S. Saturated heterocyclyl radicals are preferred.

As used herein, the term halogen atom refers typically to chlorine, fluorine, bromine and iodine atoms, preferably fluorine, chlorine and bromine atoms. The term halo when used as a prefix has the same meaning.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic, cyclohexylsulfamic (cyclamic) or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X⁻) is associated with the positive charge on the N atom. X⁻ may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X⁻ is preferably an anion selected from chloride, bromide, iodide, sulphate, nitrate, acetate, maleate, oxalate, succinate or trifluoroacetate. More preferably X⁻ is chloride, bromide, trifluoroacetate or methanesulphonate.

In the particular case where R³ is a COOH group, it is advantageous to have salts derived from the corresponding carboxylic acid by replacement of the hydrogen atom of the carboxylic group with a cation derived from a pharmaceutically acceptable base as described above.

As used herein, an N-oxide is formed from the tertiary basic amines or imines present in the molecule, using a convenient oxidising agent.

Typically, R¹ is selected from the group consisting of hydrogen atoms, fluorine atoms, chlorine atoms, bromine atoms, C₁₋₄ alkyl, C₃₋₄ cycloalkyl and —CF₃ groups.

Typically R² is selected from the group consisting of hydrogen atoms, halogen atoms and methyl groups.

Typically, G¹ is selected from the group consisting of nitrogen atoms, CCl, CF, CH, C(CH₃), C(cyclopropyl), C(phenyl) and C(CF₃) groups.

Typically G² represents a group selected from:
a hydrogen atom, a halogen atom, a C₃₋₄ cycloalkyl group, a C₁₋₂ alkoxy group and —NR^{a}R^{b}, wherein
   R^{a} represents a C₁₋₂ alkyl group and R^{b} is selected from the group consisting of C₁₋₂ alkyl groups and C₁₋₂ alkoxy-C₁₋₂ alkyl groups, or
   Ra and R^{b} together with the nitrogen atom to which they are attached form a saturated 6 or 7 membered heterocyclic ring optionally containing one oxygen atom as an additional heteroatom,
a monocyclic or bicyclic 5 to 10 membered heteroaromatic ring containing one or two nitrogen atoms which is optionally substituted by one or more substituents selected from halogen atoms and C₁₋₄ alkyl groups,
and
a phenyl group which is optionally substituted by one, two or three substituents selected from halogen atoms, C₁₋₄ alkyl, hydroxyl, C₁₋₄ alkoxy, C₃₋₄cycloalkyl, C₃₋₄ cycloalkoxy, cyano, —CF₃, —OCF₃, —CONR⁷R⁸ and oxadiazolyl groups, which oxadiazolyl groups are optionally substituted by a C₁₋₄ alkyl or a C₃₋₇ cycloalkyl group and wherein R⁷ and R⁸ are independently selected from hydrogen atoms, linear or branched C₁₋₄ alkyl groups, C₃₋₄ cycloalkyl groups, or R⁷ and R⁸ together with the nitrogen atom to which they are attached form a group of formula

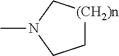

wherein n is 1 or 2,
or, when G′ represents CR⁶, G² together with R⁶ forms a non-aromatic C₆ carbocyclic group or a phenyl group.

More typically G² represents a group selected from:
a hydrogen atom, a fluorine atom, a cyclopropyl group, a methoxy group, —NMeEt, —NEt₂, —N(Me)-(CH₂)₂—O—CH₃, 6-morpholinyl, azepan-1-yl and piperidin-1-yl,
a pyridinyl, pyrimidinyl, quinolinyl or pyrazinyl ring optionally substituted with one or two substituents selected from Me and F
and
a phenyl group which is optionally substituted by one, two or three substituents selected from fluorine, chlorine, methyl, hydroxyl, methoxy, ethoxy, isopropyloxy, cyclopropyl, cyclopropyloxy, cyano, —CF₃, —OCF₃, oxadiazolyl and —CONR⁷R⁸ groups, which oxadiazolyl groups are optionally substituted by a methyl group and wherein R⁷ and R⁸ are independently selected from hydrogen atom, methyl group, isopropyl group, cyclopropyl group, or R⁷ and R⁸ together with the nitrogen atom to which they are attached form a group of formula

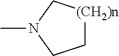

wherein n is 1,
   or, when G′ represents CR⁶, G² together with R⁶ forms a non-aromatic C₆ carbocyclic group or a phenyl group.

Even more typically, G² represents a group selected from methoxy group, cyclopropyl group and optionally substituted phenyl, pyridyl, quinolynyl, pyrimidinyl and pyrazinyl groups.

In one embodiment of the present invention, R⁹ represents a hydrogen atom, and G² represents a group selected from:
a monocyclic or bicyclic 5 to 10 membered heteroaromatic ring containing a nitrogen atom which is optionally substituted by one or more substituents selected from halogen atoms, C₁₋₄ alkyl, C₁₋₄ alkoxy, C₃₋₄ cycloalkyl, C₃₋₄ cycloalkoxy, —CF₃, —OCF₃, and —CONR⁷R⁸, wherein R⁷ and R⁸ are independently selected from hydrogen atom, lineal or branched C₁₋₄ alkyl group, C₃₋₇ cycloalkyl group, or R⁷ and R⁸ together with the nitrogen atom to which they are attached form a group of formula

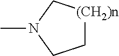

wherein n is an integer from 0 to 3,
and
a phenyl group which is optionally substituted by one or more substituents selected from halogen atoms, C₁₋₄ alkyl, C₁₋₄ alkoxy, C₃₋₄cycloalkyl, C₃₋₄ cycloalkoxy, —CF₃, —OCF₃, —CONR⁷R⁸, oxadiazolyl, triazolyl, pyrazolyl and imidazolyl groups, wherein the oxadiazolyl, triazolyl, pyrazolyl and imidazolyl groups are optionally substituted by C₁₋₄ alkyl or C₃₋₇ cycloalkyl group and wherein R⁷ and R⁸ are independently selected from hydrogen atom, lineal or branched C₁₋₄ alkyl group, a C₃₋₇ cycloalkyl group, or R⁷ and R⁸ together with the nitrogen atom to which they are attached form a group of formula

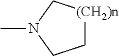

wherein n is an integer from 0 to 3
   and the pharmaceutically acceptable salts and N-oxides thereof.

Typically, R¹ is selected from the group consisting of C₁₋₄ alkyl, C₃₋₄ cycloalkyl and —CF₃, preferably methyl and cyclopropyl group, more preferably a cyclopropyl group.

Typically, R² is selected from a hydrogen or halogen atom, preferably a hydrogen atom.

Typically, R³ is selected from COOR⁵, —CONHR⁵ and tetrazolyl group; preferably R³ is a COOH group.

Typically, R⁴ represents a hydrogen atom or a methyl group, preferably a hydrogen atom.

Typically, R⁹ represents a hydrogen atom.

Typically, G¹ represents a group selected from N, CH, C(CH₃), C(cyclopropyl), C(phenyl) or C(CF₃) groups.

Typically, G² is selected from the group consisting of a methoxy group, a cyclopropyl group and optionally substituted phenyl, pyridyl, quinolynyl, pyrimidinyl and pyrazinyl groups, more preferably, G² is selected from the group consisting of optionally substituted phenyl, pyridyl, quinolynyl, pyrimidinyl and pyrazinyl groups, most preferably selected from optionally substituted phenyl, 4-pyridyl, 5-quinolynyl and 2-pyrazinyl groups.

In yet another embodiment of the present invention, R¹ is selected from a methyl or cyclopropyl group, R² represents a hydrogen atom, R³ is a COOH group, R⁴ represents a hydrogen atom or a methyl group, G¹ is selected from N, CH, C(CH₃), C(cyclopropyl), C(phenyl) and C(CF₃) groups, and G² represents a group selected from the group consisting of optionally substituted phenyl, 4-pyridyl, 5-quinolynyl and 2-pyrazinyl groups, more preferably R⁹ represent a hydrogen group.

In yet another embodiment of the present invention, R¹ is selected from a methyl or cyclopropyl group, R² represents a hydrogen atom, R³ is a COOH group, R⁴ represents a hydrogen atom, G¹ is selected from nitrogen atoms and CH, C(CH₃) and C(CF₃) groups and G² represents a phenyl group optionally substituted with one or two substituents selected from chloro, fluoro, methoxy, ethoxy, isopropoxy, trifluoromethoxy and —CONR⁷R⁸, wherein R⁷ is hydrogen and R⁸ is cyclopropyl or R⁷ and R⁸ together with the nitrogen atom to which they are attached form a group of formula

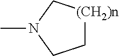

wherein n is 1.

Preferably, the DHODH inhibitor is one of the following list:
1. 5-cyclopropyl-2-(2-phenylpyrimidin-5-ylamino)benzoic acid;
2. 2-(6-Cyclopropyl-5-methylpyridin-3-ylamino)-5-methylbenzoic acid;
3. 5-(2-Carboxy-4-cyclopropylphenylamino)-3-methyl-2-phenylpyridine 1-oxide;
4. 5-Methyl-2-(6-(3-(trifluoromethyl)phenyl)pyridin-3-ylamino)benzoic acid;
5. 5-cyclopropyl-2-(6-hydroxy-5-phenylpyridin-3-ylamino)benzoic acid;
6. 5-cyclopropyl-2-(2-(2,6-difluoro-4-hydroxyphenyl)pyrimidin-5-ylamino)benzoic acid;
7. 5-Cyclopropyl-2-(6-methoxy-5-phenylpyridin-3-ylamino)benzoic acid;
8. 2-(5-Fluoro-6-phenylpyridin-3-ylamino)-5-methylbenzoic acid;
9. 2-(6-(Ethyl(methyl)amino)-5-methylpyridin-3-ylamino)-5-methylbenzoic acid;
10. 5-Cyclopropyl-2-(3′-fluoro-2,4′-bipyridin-5-ylamino)benzoic acid;

11. 2-(6-(Diethylamino)-5-methylpyridin-3-ylamino)-5-methylbenzoic acid;
12. 2-(6-((2-Methoxyethyl)(methyl)amino)-5-methylpyridin-3-ylamino)-5-methylbenzoic acid;
13. 2-(5-Chloro-6-phenylpyridin-3-ylamino)-5-methylbenzoic acid;
14. 5-Cyclopropyl-2-(2-(2-cyclopropylphenyl)pyrimidin-5-ylamino)benzoic acid;
15. 5-cyclopropyl-2-(5-phenylpyridin-3-ylamino)benzoic acid;
16. 5-methyl-2-(quinolin-3-ylamino)benzoic acid;
17. 5-methyl-2-(5,6,7,8-tetrahydroquinolin-3-ylamino)benzoic acid;
18. 2-(5-Chloro-2-phenylpyridin-3-ylamino)-5-methylbenzoic acid;
19. 5-Cyclopropyl-2-(5,6-diphenylpyridin-3-ylamino)benzoic acid;
20. 5-cyclopropyl-2-(2-(2,6-difluorophenyl)pyrimidin-5-ylamino)benzoic acid;
21. 5-Cyclopropyl-2-(5-methylpyridin-3-ylamino)benzoic acid;
22. 2-(2-(3-Cyclopropoxyphenyl)pyrimidin-5-ylamino)-5-cyclopropylbenzoic acid;
23. 5-Methyl-2-(6-morpholinopyridin-3-ylamino)benzoic acid;
24. 5-Methyl-2-(5-methyl-6-morpholinopyridin-3-ylamino)benzoic acid;
25. 5-cyclopropyl-2-(6-cyclopropyl-5-phenylpyridin-3-ylamino)benzoic acid;
26. 2-(6-(2-Cyclopropylphenyl)-5-methylpyridin-3-ylamino)-5-methylbenzoic acid;
27. 2-(6-(2-Cyanophenyl)-5-methylpyridin-3-ylamino)-5-methylbenzoic acid;
28. 2-(2-(3-Chlorophenyl)pyrimidin-5-ylamino)-5-cyclopropylbenzoic acid;
29. 5-Methyl-2-(6-phenyl-5-(trifluoromethyl)pyridin-3-ylamino)benzoic acid;
30. 5-Methyl-2-(5-methyl-6-(piperidin-1-yl)pyridin-3-ylamino)benzoic acid;
31. 2-(6-(Azepan-1-yl)-5-methylpyridin-3-ylamino)-5-methylbenzoic acid;
32. 2-(6-(3-Methoxyphenyl)-5-phenylpyridin-3-ylamino)-5-methylbenzoic acid;
33. 2-(2,3′-bipyridin-5-ylamino)-5-cyclopropylbenzoic acid;
34. 2-(3′-chloro-2,4′-bipyridin-5-ylamino)-5-methylbenzoic acid;
35. 5-Methyl-2-(3-methyl-2,2′-bipyridin-5-ylamino)benzoic acid;
36. 2-(5,6-Difluoropyridin-3-ylamino)-5-methylbenzoic acid;
37. 2-(6-(3-Methoxyphenyl)pyridin-3-ylamino)benzoic acid;
38. 2-(6-(3-Ethoxyphenyl)pyridin-3-ylamino)benzoic acid;
39. 2-(6-(3-Ethoxyphenyl)pyridin-3-ylamino)-5-fluorobenzoic acid;
40. 2-(6-(3-Ethoxyphenyl)-5-methylpyridin-3-ylamino)benzoic acid;
41. 2-(6-(3-Ethoxyphenyl)pyridin-3-ylamino)-5-methylbenzoic acid;
42. 2-(6-(3-Ethoxyphenyl)-5-methylpyridin-3-ylamino)-5-methylbenzoic acid;
43. 2-(6-(3-Ethoxy-2-fluorophenyl)pyridin-3-ylamino)benzoic acid;
44. 2-(6-(3-Ethoxyphenyl)-4-methylpyridin-3-ylamino)-5-methylbenzoic acid;
45. 2-(6-(3-Ethoxyphenyl)-4-methylpyridin-3-ylamino)benzoic acid;
46. 5-Bromo-2-(6-(3-ethoxyphenyl)pyridin-3-ylamino)benzoic acid;
47. 5-Chloro-2-(6-(3-ethoxyphenyl)pyridin-3-ylamino)benzoic acid;
48. 2-(6-(5-Ethoxy-2-fluorophenyl)pyridin-3-ylamino)benzoic acid;
49. 2-(6-(3-Ethoxyphenyl)-5-methylpyridin-3-ylamino)-5-(trifluoromethyl)benzoic acid;
50. 2-(6-(3-Methoxyphenyl)-5-methylpyridin-3-ylamino)-5-(trifluoromethyl)benzoic acid;
51. 2-(6-(3-Methoxyphenyl)-5-methylpyridin-3-ylamino)-5-methylbenzoic acid;
52. 2-(6-(3-Methoxyphenyl)-5-methylpyridin-3-ylamino)-6-methylbenzoic acid;
53. 5-Fluoro-2-(6-(3-methoxyphenyl)-5-methylpyridin-3-ylamino)benzoic acid;
54. 2-(6-(5-Ethoxy-2-fluorophenyl)pyridin-3-ylamino)-5-methylbenzoic acid;
55. 2-(6-(2-Fluoro-5-methoxyphenyl)-5-methylpyridin-3-ylamino)-5-methylbenzoic acid;
56. Ethyl 2-(6-(2-fluoro-5-methoxyphenyl)-5-methylpyridin-3-ylamino)-5-methylbenzoate;
57. 2-(6-(2-Fluorophenyl)pyridin-3-ylamino)-5-methylbenzoic acid;
58. 2-(6-(3-Methoxyphenyl)-5-phenylpyridin-3-ylamino)-5-methylbenzoic acid;
59. Ethyl 2-(6-(3-methoxyphenyl)-5-phenylpyridin-3-ylamino)-5-methylbenzoate;
60. 5-Methyl-2-(5-methyl-6-phenylpyridin-3-ylamino)benzoic acid;
61. Ethyl 5-methyl-2-(5-methyl-6-phenylpyridin-3-ylamino)benzoate;
62. 5-Methyl-2-(5-methyl-6-(3-(trifluoromethoxy)phenyl)pyridin-3-ylamino)benzoic acid;
63. Ethyl 5-methyl-2-(5-methyl-6-(3-(trifluoromethoxy)phenyl)pyridin-3-ylamino)benzoate;
64. 2-(5-Cyclopropyl-6-(3-methoxyphenyl)pyridin-3-ylamino)-5-methyl benzoic acid;
65. Ethyl 2-(5-cyclopropyl-6-(3-methoxyphenyl)pyridin-3-ylamino)-5-methylbenzoate;
66. 2-(6-(2-Fluoro-5-isopropoxyphenyl)pyridin-3-ylamino)-5-methylbenzoic acid;
67. 2-(6-(3-Isopropoxyphenyl)-5-methylpyridin-3-ylamino)-5-methylbenzoic acid;
68. Ethyl 2-(6-(3-isopropoxyphenyl)-5-methylpyridin-3-ylamino)-5-methylbenzoate;
69. 2-(6-(3-Cyclopropoxyphenyl)-5-methylpyridin-3-ylamino)-5-methylbenzoic acid;
70. tert-Butyl 2-(6-(3-cyclopropoxyphenyl)-5-methylpyridin-3-ylamino)-5-methylbenzoate;
71. 2-(6-(2-Chlorophenyl)-5-methylpyridin-3-ylamino)-5-methylbenzoic acid;
72. tert-Butyl 2-(6-(2-chlorophenyl)-5-methylpyridin-3-ylamino)-5-methylbenzoate;
73. 2-(6-(3-Carbamoylphenyl)-5-methylpyridin-3-ylamino)-5-methylbenzoic acid;
74. Ethyl 2-(6-(3-carbamoylphenyl)-5-methylpyridin-3-ylamino)-5-methylbenzoate;
75. 2-(6-(2-Fluoro-5-methoxyphenyl)-4-methylpyridin-3-ylamino)-5-methylbenzoic acid;
76. Ethyl 2-(6-(2-fluoro-5-methoxyphenyl)-4-methylpyridin-3-ylamino)-5-methylbenzoate;
77. 2-(6-(3-Methoxyphenyl)-5-(trifluoromethyl)pyridin-3-ylamino)-5-methylbenzoic acid;
78. Ethyl 2-(6-(3-methoxyphenyl)-5-(trifluoromethyl)pyridin-3-ylamino)-5-methylbenzoate;
79. 2-(6-(3-(Dimethylcarbamoyl)phenyl)-5-methylpyridin-3-ylamino)-5-methylbenzoic acid;
80. Ethyl 2-(6-(3-(dimethylcarbamoyl)phenyl)-5-methylpyridin-3-ylamino)-5-methylbenzoate;
81. 2-(6-(3-Isopropoxyphenyl)-5-methylpyridin-3-ylamino)-3-methylbenzoic acid;
82. tert-Butyl 2-(6-(3-isopropoxyphenyl)-5-methylpyridin-3-ylamino)-3-methyl benzoate;
83. 3-Methyl-2-(5-methyl-6-phenylpyridin-3-ylamino)benzoic acid;
84. tert-Butyl 3-methyl-2-(5-methyl-6-phenylpyridin-3-ylamino)benzoate;
85. 2-(6-(2-Chlorophenyl)pyridin-3-ylamino)-5-methylbenzoic acid;
86. tert-Butyl 2-(6-(2-chlorophenyl)pyridin-3-ylamino)-5-methylbenzoate;
87. 3-Fluoro-2-(6-(3-methoxyphenyl)-5-methylpyridin-3-ylamino)benzoic acid;
88. tert-Butyl 3-fluoro-2-(6-(3-methoxyphenyl)-5-methylpyridin-3-ylamino)benzoate;
89. 5-Cyclopropyl-2-(5-methyl-6-(3-(trifluoromethoxy)phenyl)pyridin-3-ylamino)benzoic acid;
90. Ethyl 5-cyclopropyl-2-(5-methyl-6-(3-(trifluoromethoxy)phenyl)pyridin-3-ylamino)benzoate;
91. 5-Cyclopropyl-2-(5-methyl-6-phenylpyridin-3-ylamino)benzoic acid;
92. Ethyl 5-cyclopropyl-2-(5-methyl-6-phenylpyridin-3-ylamino)benzoate;
93. 5-Methyl-2-(5-methyl-6-(2-(trifluoromethyl)phenyl)pyridin-3-ylamino)benzoic acid;
94. tert-Butyl 5-methyl-2-(5-methyl-6-(2-(trifluoromethyl)phenyl)pyridin-3-ylamino)benzoate;
95. 2-(6-(3-Chlorophenyl)-5-methylpyridin-3-ylamino)-5-methylbenzoic acid;
96. tert-Butyl 2-(6-(3-chlorophenyl)-5-methylpyridin-3-ylamino)-5-methylbenzoate;
97. 2-(6-(2-Fluorophenyl)-5-methylpyridin-3-ylamino)-5-methylbenzoic acid;
98. tert-Butyl 2-(6-(2-fluorophenyl)-5-methylpyridin-3-ylamino)-5-methylbenzoate;
99. 5-Methyl-2-(5-methyl-6-(quinolin-5-yl)pyridin-3-ylamino)benzoic acid;
100. tert-Butyl 5-methyl-2-(5-methyl-6-(quinolin-5-yl)pyridin-3-ylamino)benzoate;
101. 2-(3′-Fluoro-3-methyl-2,4′-bipyridin-5-ylamino)-5-methylbenzoic acid;
102. tert-Butyl 2-(3′-fluoro-3-methyl-2,4′-bipyridin-5-ylamino)-5-methylbenzoate;
103. 5-Methyl-2-(5-methyl-6-(pyrazin-2-yl)pyridin-3-ylamino)benzoic acid;
104. tert-Butyl 5-methyl-2-(5-methyl-6-(pyrazin-2-yl)pyridin-3-ylamino)benzoate;
105. 5-Cyclopropyl-2-(6-phenyl-5-(trifluoromethyl)pyridin-3-ylamino)benzoic acid;
106. Ethyl 5-cyclopropyl-2-(6-phenyl-5-(trifluoromethyl)pyridin-3-ylamino)benzoate;
107. 5-Cyclopropyl-2-(6-(3-methoxyphenyl)-5-(trifluoromethyl)pyridin-3-ylamino)benzoic acid;
108. Ethyl 5-cyclopropyl-2-(6-(3-methoxyphenyl)-5-(trifluoromethyl)pyridin-3-ylamino)benzoate;
109. 5-Chloro-2-(6-(2-fluorophenyl)pyridin-3-ylamino)benzoic acid;
110. 5-Chloro-2-(6-(2-chlorophenyl)pyridin-3-ylamino)benzoic acid;
111. 5-Chloro-2-(6-(quinolin-5-yl)pyridin-3-ylamino)benzoic acid;
112. 2-(6-(2-Chlorophenyl)pyridin-3-ylamino)-5-cyclopropylbenzoic acid;
113. Ethyl 2-(6-(2-chlorophenyl)pyridin-3-ylamino)-5-cyclopropylbenzoate;
114. 5-Chloro-2-(6-(2-(trifluoromethyl)phenyl)pyridin-3-ylamino)benzoic acid;
115. 5-Fluoro-2-(6-(2-(trifluoromethyl)phenyl)pyridin-3-ylamino)benzoic acid;
116. 2-(3′-Fluoro-2,4′-bipyridin-5-ylamino)-5-methylbenzoic acid;
117. 2-(2-(2-Fluorophenyl)pyrimidin-5-ylamino)-5-methylbenzoic acid;
118. tert-Butyl 2-(2-(2-fluorophenyl)pyrimidin-5-ylamino)-5-methylbenzoate;
119. 2-(6-(2,6-Difluorophenyl)pyridin-3-ylamino)-5-methylbenzoic acid;
120. Ethyl 2-(6-(2,6-difluorophenyl)pyridin-3-ylamino)-5-methylbenzoate;
121. 2-(2-(2-Chlorophenyl)pyrimidin-5-ylamino)-5-cyclopropylbenzoic acid;
122. Methyl 2-(2-(2-chlorophenyl)pyrimidin-5-ylamino)-5-cyclopropylbenzoate;
123. 2-(2-(2-Chlorophenyl)pyrimidin-5-ylamino)-5-methylbenzoic acid;
124. tent-Butyl 2-(2-(2-chlorophenyl)pyrimidin-5-ylamino)-5-methylbenzoate;
125. 5-Methyl-2-(5-methyl-6-(3-(pyrrolidine-1-carbonyl)phenyl)pyridin-3-ylamino)benzoic acid;
126. 2-(6-(3-(Cyclopropylcarbamoyl)phenyl)-5-methylpyridin-3-ylamino)-5-methylbenzoic acid;
127. 5-Cyclopropyl-2-(2-(2-fluorophenyl)pyrimidin-5-ylamino)benzoic acid;
128. 2-(2-(2-trifluoromethylphenyl)pyrimidin-5-ylamino)-5-cyclopropylbenzoic acid;
129. 2-(2-o-tolylpyrimidin-5-ylamino)-5-cyclopropylbenzoic acid;
130. 2-(2-(2-cyclopropoxyphenyl)pyrimidin-5-ylamino)-5-cyclopropylbenzoic acid;
131. 2-(2-(2,5-difluorophenyl)pyrimidin-5-ylamino)-5-cyclopropylbenzoic acid;
132. 2-(2-(2,3-difluorophenyl)pyrimidin-5-ylamino)-5-cyclopropylbenzoic acid;
133. 2-(2-(2-fluoro-5-chlorophenyl)pyrimidin-5-ylamino)-5-cyclopropylbenzoic acid;
134. 2-(2-(2-trifluoromethylphenyl)pyrimidin-5-ylamino)-5-methylbenzoic acid;
135. 2-(2-(2-fluoro-5-trifluoromethoxyphenyl)pyrimidin-5-ylamino)-5-cyclopropylbenzoic acid;
136. 2-(6-(2-trifluoromethylphenyl)pyridin-3-ylamino)-5-methylbenzoic acid;
137. 2-(6-phenylpyridin-3-ylamino)-5-cyclopropylbenzoic acid;
138. 2-(6-(2-fluorophenyl)pyridin-3-ylamino)-5-cyclopropylbenzoic acid;
139. 2-(6-(3,5-difluoropyridin-4-yl)pyridin-3-ylamino)-5-methylbenzoic acid;
140. 2-(6-(3-cyclopropylcarbamoylphenyl)pyridin-3-ylamino)-5-methylbenzoic acid;
141. 2-(6-(2,4-difluorophenyl)pyridin-3-ylamino)-5-methylbenzoic acid;
142. 2-(6-(2,5-difluorophenyl)pyridin-3-ylamino)-5-methylbenzoic acid;
143. 2-(6-(2-fluorophenyl)pyridin-3-ylamino)-5-cyclopropyl-3-fluorobenzoic acid;
144. 2-(6-(2,3,6-trifluorophenyl)pyridin-3-ylamino)-5-methylbenzoic acid;
145. 2-(6-(3-(5-methyl-1,3,4-oxadiazol-2-yl)phenyl)pyridin-3-ylamino)-5-methylbenzoic acid;
146. 2-(5-methyl-6-(pyrimidin-5-yl)pyridin-3-ylamino)-5-methylbenzoic acid;
147. 2-(6-(2,3-difluorophenyl)pyridin-3-ylamino)-5-methylbenzoic acid;
148. 2-(6-(5-fluoro-2-methoxyphenyl)-5-methylpyridin-3-ylamino)-5-methylbenzoic acid, and;
149. 2-(6-(4-carbamoylphenyl)-5-methylpyridin-3-ylamino)-5-methylbenzoic acid;
   or a pharmaceutically acceptable salt or N-oxide thereof.

More preferably, the DHODH inhibitor is one of:
5-cyclopropyl-2-(2-phenylpyrimidin-5-ylamino)benzoic acid;
5-(2-Carboxy-4-cyclopropylphenylamino)-3-methyl-2-phenylpyridine 1-oxide;
5-Methyl-2-(6-(3-(trifluoromethyl)phenyl)pyridin-3-ylamino)benzoic acid;
2-(5-Fluoro-6-phenylpyridin-3-ylamino)-5-methylbenzoic acid;
5-Cyclopropyl-2-(3′-fluoro-2,4′-bipyridin-5-ylamino)benzoic acid;
2-(5-Chloro-6-phenylpyridin-3-ylamino)-5-methylbenzoic acid;
5-Cyclopropyl-2-(2-(2-cyclopropylphenyl)pyrimidin-5-ylamino)benzoic acid;
5-cyclopropyl-2-(5-phenylpyridin-3-ylamino)benzoic acid;
2-(5-Chloro-2-phenylpyridin-3-ylamino)-5-methylbenzoic acid;
5-Cyclopropyl-2-(5,6-diphenylpyridin-3-ylamino)benzoic acid;
5-cyclopropyl-2-(2-(2,6-difluorophenyl)pyrimidin-5-ylamino)benzoic acid;
2-(2-(3-Cyclopropoxyphenyl)pyrimidin-5-ylamino)-5-cyclopropylbenzoic acid;
5-Methyl-2-(6-morpholinopyridin-3-ylamino)benzoic acid;
5-Methyl-2-(5-methyl-6-morpholinopyridin-3-ylamino)benzoic acid;
5-cyclopropyl-2-(6-cyclopropyl-5-phenylpyridin-3-ylamino)benzoic acid;
2-(6-(2-Cyclopropylphenyl)-5-methylpyridin-3-ylamino)-5-methylbenzoic acid;
2-(6-(2-Cyanophenyl)-5-methylpyridin-3-ylamino)-5-methylbenzoic acid;
2-(2-(3-Chlorophenyl)pyrimidin-5-ylamino)-5-cyclopropylbenzoic acid;
5-Methyl-2-(6-phenyl-5-(trifluoromethyl)pyridin-3-ylamino)benzoic acid;
5-Methyl-2-(5-methyl-6-(piperidin-1-yl)pyridin-3-ylamino)benzoic acid;
2-(6-(Azepan-1-yl)-5-methylpyridin-3-ylamino)-5-methylbenzoic acid;
2-(2,3′-bipyridin-5-ylamino)-5-cyclopropylbenzoic acid;
2-(3′-chloro-2,4′-bipyridin-5-ylamino)-5-methylbenzoic acid;
5-Methyl-2-(3-methyl-2,2′-bipyridin-5-ylamino)benzoic acid;
2-(6-(3-Ethoxyphenyl)-5-methylpyridin-3-ylamino)-5-methylbenzoic acid;
2-(6-(3-Methoxyphenyl)-5-methylpyridin-3-ylamino)-5-methylbenzoic acid;
5-Methyl-2-(5-methyl-6-phenylpyridin-3-ylamino)benzoic acid;
2-(6-(3-Isopropoxyphenyl)-5-methylpyridin-3-ylamino)-5-methylbenzoic acid;
5-Cyclopropyl-2-(5-methyl-6-(3-(trifluoromethoxy)phenyl)pyridin-3-ylamino)benzoic acid;
5-Cyclopropyl-2-(5-methyl-6-phenylpyridin-3-ylamino)benzoic acid;
2-(6-(2-Fluorophenyl)-5-methylpyridin-3-ylamino)-5-methylbenzoic acid;
5-Cyclopropyl-2-(6-phenyl-5-(trifluoromethyl)pyridin-3-ylamino)benzoic acid;
5-Cyclopropyl-2-(6-(3-methoxyphenyl)-5-(trifluoromethyl)pyridin-3-ylamino)benzoic acid;
2-(6-(2-Chlorophenyl)pyridin-3-ylamino)-5-cyclopropylbenzoic acid;
2-(2-(2-Chlorophenyl)pyrimidin-5-ylamino)-5-cyclopropylbenzoic acid;
2-(2-(2-Chlorophenyl)pyrimidin-5-ylamino)-5-methylbenzoic acid;
2-(6-(2,6-Difluorophenyl)pyridin-3-ylamino)-5-methylbenzoic acid;
5-Methyl-2-(5-methyl-6-(3-(pyrrolidine-1-carbonyl)phenyl)pyridin-3-ylamino)benzoic acid;
2-(6-(3-(Cyclopropylcarbamoyl)phenyl)-5-methylpyridin-3-ylamino)-5-methylbenzoic acid;
5-Cyclopropyl-2-(2-(2-fluorophenyl)pyrimidin-5-ylamino)benzoic acid,
   or a pharmaceutically acceptable salt or N-oxide thereof.

Most preferably, the DHODH inhibitor is 5-Methyl-2-(6-(3-(trifluoromethyl)phenyl)pyridin-3-ylamino)benzoic acid, 5-cyclopropyl-2-(2-(2,6-difluorophenyl)pyrimidin-5-ylamino)benzoic acid, 2-(6-(2,6-Difluorophenyl)pyridin-3-ylamino)-5-methylbenzoic acid or 2-(6-(3-(Cyclopropylcarbamoyl)phenyl)-5-methylpyridin-3-ylamino)-5-methylbenzoic acid or a pharmaceutically acceptable salt or N-oxide thereof.

Preferably the active ingredients (a) and (b) form part of a single pharmaceutical composition.

Further provided is a combination as described above which further comprises (c) another compound selected from:
(i) Anti-TNF-alpha monoclonal antibodies such as Infliximab, Certolizumab pegol, Golimumab, Adalimumab and AME-527 from Applied Molecular Evolution;
(ii) TNF-alpha Antagonists such as Etanercept, Lenercept, Onercept and Pegsunercept;
(iii) Calcineurin (PP-2B) Inhibitors/INS Expression Inhibitors such as cyclosporine A, Tacrolimus and ISA-247 from Isotechnika;
(iv) IL-1 Receptor Antagonists such as Anakinra and AMG-719 from Amgen;
(v) Anti-CD20 monoclonal antibodies such as Rituximab, Ofatumumab, Ocrelizumab and TRU-015 from Trubion Pharmaceuticals;
(vi) p38 Inhibitors such as AMG-548 (from Amgen), ARRay-797 (from Array Biopharma), Chlormethiazole edisylate, Doramapimod, PS-540446 (from BMS), SB-203580, SB-242235, SB-235699, SB-281832, SB-681323, SB-856553 (all from GlaxoSmithKline), KC-706 (from Kemia), LEO-1606, LEO-15520 (all from Leo), SC-80036, SD-06 (all from Pfizer), RWJ-67657 (from R. W. Johnson), RO-3201195, RO-4402257 (all from Roche), AVE-9940 (from Aventis), SC10-323, SCIO-469 (all from Scios), TA-5493 (from Tanabe Seiyaku), and VX-745 and VX-702 (all from Vertex);
(vii) NF-kappaB (NFKB) Activation Inhibitors such as Sulfasalazine and Iguratimod;
(viii) Another dihydrofolate reductase (DHFR) inhibitor such as Aminopterin and CH-1504 from Chelsea;
(ix) Janus kinase (JAK) inhibitors, such as CP-690, 550 from Pfizer and INCB-18424, from Incyte;
(x) MEK inhibitor, such as ARRY-162 from Array;
(xi) Sphingosine-1 phosphate receptor agonists, such as fingolimod (Novartis);
(xii) Interferons comprising Interferon beta 1a such as Avonex from Biogen Idec, CinnoVex from CinnaGen and Rebif from Merck Serono, and Interferon beta 1b such as Betaferon from Schering and Betaseron from Berlex;
(xiii) Immunomodulators suchs as BG-12 (fumaric acid derivative) from Biogen Idec/Fumapharm AG; laquinimod (Teva and Active Biotech) or glatiramer acetate (Teva); and
(xiv) Adenosine aminohydrolase inhibitors such as Cladribine from Merck Serono.

The present invention further provides use of (a) methotrexate and (b) a DHODH inhibitor of the invention for the preparation of a medicament for simultaneous, separate or sequential use in the treatment of a pathological condition or disease susceptible to amelioration by inhibition of dehydroorotate dehydrogenase.

Diseases or disorders in which DHODH inhibition plays a role include without limitation autoimmune diseases, immune and inflammatory diseases, destructive bone disorders, malignant neoplastic diseases, angiogenic-related disorders, viral diseases, and infectious diseases.

Autoimmune diseases which may be prevented or treated include but are not limited to rheumatoid arthritis, psoriatic arthritis, systemic lupus erythematosus, multiple sclerosis, psoriasis, ankylosing spondilytis, Wegener's granulomatosis, polyarticular juvenile idiopathic arthritis, inflammatory bowel disease such as ulcerative colitis and Crohn's disease, Reiter's syndrome, fibromyalgia and type-1 diabetes.

Immune and inflammatory diseases which may be prevented or treated include but are not limited to asthma, COPD, respiratory distress syndrome, acute or chronic pancreatitis, graft versus-host disease, chronic sarcoidosis, transplant rejection, contact dermatitis, atopic dermatitis, allergic rhinitis, allergic conjunctivitis, Behcet syndrome, inflammatory eye conditions such as conjunctivitis and uveitis.

Destructive bone disorders which may be prevented or treated include but are not limited to osteoporosis, osteoarthritis and multiple myeloma-related bone disorder.

Malignant neoplastic diseases that may be prevented or treated include but are not limited to prostate, ovarian and brain cancer.

Angiogenesis-related disorders that may be prevented or treated include but are not limited to haemangioma, ocular neovascularization, macular degeneration or diabetic retinopathy.

Viral diseases which may be prevented or treated include but are not limited to HIV infection, hepatitis and cytomegalovirus infection.

Infectious diseases which may be prevented or treated include but are not limited to sepsis, septic shock, endotoxic shock, Gram negative sepsis, toxic shock syndrome, Shigellosis and other protozoal infestations such as malaria.

Preferably, the pathological condition or disease is selected from rheumatoid arthritis, psoriatic arthritis, ankylosing spondilytis, multiple sclerosis, Wegener's granulomatosis, systemic lupus erythematosus, psoriasis and sarcoidosis. More preferably the pathological condition or disease is rheumatoid arthritis, psoriatic arthritis or psoriasis. Most preferably it is rheumatoid arthritis.

Also provided is a combination comprising (a) Interferons such as Interferon beta 1a or Interferon beta 1b, and (b) a DHODH inhibitor of the invention, preferably a DHODH inhibitor of formula (I).

Also provided is the use of a combination comprising (a) Interferons such as Interferon beta 1a or Interferon beta 1b, and (b) a DHODH inhibitor of the invention, preferably a DHODH inhibitor of formula (I) for the preparation of a medicament for simultaneous, separate or sequential use for the treatment of multiple scleroris.

Also provided is a product comprising (a) methotrexate and (b) a DHODH inhibitor of the invention, as a combined preparation for simultaneous, separate or sequential use in the treatment of a human or animal patient suffering from or susceptible to a pathological condition or disease as defined above. Said product may optionally further comprise an active compound (c), as defined above.

Also provided is a kit of parts comprising (b) a DHODH inhibitor of the invention together with instructions for simultaneous, separate or sequential use in combination with (a) methotrexate, for the treatment of a human or animal patient suffering from or susceptible to a pathological condition or disease as defined above. Said kit may optionally further comprise an active compound (c), as defined above.

Also provided is a package comprising (b) a DHODH inhibitor of the invention and (a) methotrexate, for simultaneous, separate or sequential use in the treatment of a pathological condition or disease as defined above. Said package may optionally further comprise an active compound (c), as defined above.

Also provided is a use of (b) a DHODH inhibitor of the invention for the preparation of a medicament, for use in combination with (a) methotrexate, for the treatment of a pathological condition or disease as defined above.

Also provided is a use of (a) methotrexate, for the preparation of a medicament, for use in combination with (b) a DHODH inhibitor of the invention, for the treatment of a pathological condition or disease as defined above.

Also provided is a use as defined above wherein the methotrexate (a) is for administration at a dosage regime which involves administration of 0.015 to 3 mg/kg/week of methotrexate and the DHODH inhibitor (b) is for administration at a dosage regime which involves administration of 0.03 to 30 mg/kg/day of DHODH inhibitor.

Typically the medicament is for use in treating a human or animal patient suffering or susceptible to hepatic impairment or a condition that would be aggravated by hepatotoxicity. More typically, the said human or animal patient is suffering from liver fibrosis, hepatitis (typically hepatitis A to G), cirrhosis (typically caused by alcoholism) or liver cancer.

In one embodiment of the present invention, the combination, product, kit of parts or package comprises (b) a DHODH inhibitor of the invention, and (a) methotrexate, as the sole active components.

The fact that the DHODH inhibitors of the invention have reduced hepatic side effects is a finding of the invention. The present invention therefore also provides the use of a DHODH inhibitor of the invention, as defined above, in the manufacture of a medicament for use in treating or preventing a pathological condition or disease, as defined above, in a human or animal patient which is suffering from or susceptible to hepatic impairment or a condition that would be aggravated by hepatotoxicity, as defined above.

Also provided is a method of treating a human or animal patient suffering from or susceptible to a pathological condition or disease as defined above, which method comprises simultaneously, separately or sequentially administering to said human or animal patient a therapeutically effective amount of (a) methotrexate and (b) a DHODH inhibitor as defined above. Preferably in said method, (a) methotrexate and (b) the DHODH inhibitor are the sole active components.

Also provided is a method of treating a human or animal patient suffering from or susceptible to a pathological condition or disease as defined above, wherein the human or animal patient is suffering from or susceptible to hepatic impairment or a condition that would be aggravated by hepatotoxicity as defined above, which method comprises administering to said human or animal patient a therapeutically effective amount of a DHODH inhibitor as defined above.

Also provided is a combination as defined above for use in treating a pathological condition or disease as defined above.

Also provided is a DHODH inhibitor as defined above for use in treating a human or animal patient suffering from or susceptible to a pathological condition or disease as defined above, wherein the human or animal patient is suffering from or susceptible to hepatic impairment or a condition that would be aggravated by hepatotoxicity, as defined above.

The active compounds in the combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.) or by inhalation (as a dry powder, a solution, a dispersion, etc).

The active compounds in the combination may be administered together in the same pharmaceutical composition or in different compositions intended for separate, simultaneous, concomitant or sequential administration by the same or a different route.

The combinations of the invention may conveniently be presented in unit dosage form and may be prepared by any of the methods well known in the art of pharmacy.

Combinations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with flavouring or colouring agent.

Where the combination is in the form of a tablet, any pharmaceutical carrier routinely used for preparing solid formulations may be used. Examples of such carriers include magnesium stearate, talc, gelatine, acacia, stearic acid, starch, lactose and sucrose.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the combination is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

The combination may be in the form of a dry powder composition for topical delivery to the lung by inhalation. Dry powder compositions may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 2 μg and 150 μg of each therapeutically active ingredient. Alternatively, the active ingredient (s) may be presented without excipients.

Packaging of the formulation for inhalation may be carried out by using suitable inhaler devices such as the Genuair® (formerly known as Novolizer SD2FL) which is described in the following patent applications: WO 97/000703, WO 03/000325 and WO 03/061742.

The combination may be in the form of a composition for nasal delivery. Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the combination is in unit dosage form, for example a tablet, capsule or metered aerosol dose, so that the patient may administer a single dose.

The amount of each active which is required to achieve a therapeutic effect will, of course, vary with the particular active, the route of administration, the subject under treatment, and the particular disorder or disease being treated.

Typically all active agents in the combination are administered at the same time, or very close in time. Alternatively, one or two actives could be taken in the morning and the other (s) later in the day. Or in another scenario, one or two actives could be taken twice daily and the other (s) once daily, either at the same time as one of the twice-a-day dosing occurred, or separately. Preferably at least two, and more preferably all, of the actives would be taken together at the same time. Preferably, at least two, and more preferably all actives would be administered as an admixture.

Preferably the drug combination of the invention is for administration as a dosage regime which involves administration of (i) 0.015 to 3 mg/kg/week of methotrexate, more preferably 0.07 to 0.7 mg/kg/week of methotrexate and most preferably 0.15 to 0.35 mg/kg/week of methotrexate, and (ii) 0.03 to 30 mg/kg/day of DHODH inhibitor, more preferably 0.07 to 14 mg/day of DHODH inhibitor and most preferably 0.15 to 0.3 mg/kg/day of DHODH inhibitor.

### EXAMPLES

### Example 1

### Inhibition of Human DHODH Activity Assay

DHODH activity and its inhibition were studied using a chromogen reduction assay with DCIP (2,6-dichlorophenol-indophenol). The substrate oxidation (Dihydroorotate, L-DHO), as well as co-substrate reduction (coenzyme Q, CoQ) is coupled to the chromogen reduction, hence enzymatic activity results in a loss of chromogen absorbance at 600 nm.

Enzyme extracts (8 μl, ˜1.5 μg of human protein) were incubated in 96-well plates. The assay mixture (200 μl) contained 200 μM CoQD, 100 μM L-DHO, 120 μM DCIP in the assay buffer (100 mM HEPES pH 8.0, 150 mM NaCl, 10% Glicerol, 0.05% Triton X-100) and 2 μl of test compound. The compounds were dissolved in DMSO at a stock concentration of 1 mM, and tested at different concentrations varying from 10 μM to 1 pM to calculate an IC₅₀ (concentration of inhibitor required for 50% of inhibition).

The reaction was initiated by adding the enzyme and then incubated for 10 min at room temperature before measuring DCIP reduction by counting a decrease in absorbance at 600 nm using standard instrumentation (Spectramax).

All reactions were carried out in duplicate and graphs, determining IC₅₀ values for each compound, were plotted using the ABase software.

Table 1 shows the activities in human DHODH inhibition assay of some compounds of the present invention (compounds from the list indicated previously) showing that these compounds are potent DHODH inhibitors.

TABLE 1

| Compound No. | hDHODH IC₅₀ (nM) |
|---|---|
| 1 | 105 |
| 4 | 98 |
| 6 | 57 |
| 7 | 49 |
| 8 | 18 |
| 14 | 57 |
| 15 | 113 |
| 18 | 62 |
| 19 | 10 |
| 20 | 14 |
| 22 | 114 |
| 25 | 28 |
| 30 | 41 |
| 31 | 119 |
| 34 | 109 |
| 41 | 190 |
| 42 | 30 |
| 44 | 78 |
| 50 | 138 |
| 51 | 21 |
| 54 | 19 |
| 57 | 91 |
| 60 | 53 |
| 66 | 28 |
| 67 | 11 |
| 71 | 14 |
| 73 | 190 |
| 75 | 97 |
| 77 | 12 |
| 79 | 33 |
| 85 | 32 |
| 89 | 5 |
| 91 | 6 |
| 93 | 20 |
| 95 | 10 |
| 97 | 5 |
| 101 | 37 |
| 105 | 2 |
| 107 | 7 |
| 110 | 145 |
| 112 | 4 |
| 116 | 90 |
| 119 | 19 |
| 121 | 3 |
| 123 | 57 |
| 125 | 9 |
| 126 | 12 |
| 127 | 10 |
| 128 | 9 |
| 129 | 12 |
| 131 | 38 |
| 135 | 21 |
| 138 | 8 |
| 141 | 146 |
| 144 | 77 |
| 148 | 46 |

### Example 2

### Reduced Hepatotoxicity

Acute hepatotoxicity assays were performed in Swiss mice. Animals received a single administration of either vehicle, or 100 mg/kg of teriflunomide or a compound of the present invention (compounds from the list indicated previously) by intraperitoneal route. Twenty-four hours later, animals were sacrificed and the levels of liver markers AST (aspartate aminotransferase), ALT (alanine aminotransferase) and BIL (total bilirubin) in plasma were determined.

TABLE 2
Plasma levels of liver markers of mice after administration of 100 mg/kg
of the compound, 100 mg/kg Teriflunomide or vehicle
(IU: International Units).

| Compound No. | ALT (IU/I) | AST (IU/I) | BIL (mg/dl) |
|---|---|---|---|
| 20 | 45 | 98 | 0.07 |
| 60 | 70 | 131 | 0.09 |
| 71 | 72 | 95 | 0.05 |
| 85 | 43 | 83 | 0.13 |
| 97 | 55 | 92 | 0.11 |
| 119 | 69 | 96 | 0.08 |
| 121 | 75 | 105 | 0.05 |
| 123 | 89 | 113 | 0.06 |
| 127 | 56 | 72 | 0.07 |
| Vehicle | 68 | 92 | 0.1 |
| Teriflunomide | 440 | 655 | 0.46 |

As it can clearly seen from Table 2, Teriflunomide-treated mice showed a dramatic increase in the three liver markers compared to vehicle-treated mice, clearly indicating a high hepatotoxicity, whereas the DHODH inhibitors according to the present invention did not cause a significant increase in any of the parameters measured

### Example 3

### Efficacy Assay in Adjuvant-Induced Arthritis of the Combination Product of the Present Invention

The effect of DHODH inhibitor compounds were tested in combination with methotrexate (0.05 mg/Kg/day) in the rat adjuvant-induced arthritis model (AIA) in animals with established disease (curative protocol). Briefly, Complete Freund Adjuvant (CFA) was injected into the left hind footpad of Wistar rats, and 10 days later the swelling of the two rear paws was measured with a plethysnnometer. Rats exhibiting a similar degree of inflammation in both paws were randomized into treatment groups (n=7 per group). Compounds were administered orally once a day for 10 days and paw volumes were determined every two days up to day 21.

TABLE 3
Effects of compound 20 (10 mg/Kg/day), Methotrexate (0.05 mg/Kg/day)
and their combination on the inhibition of paw inflammation in
arthritic rats.

| | % inhibition of the inflammation |
|---|---|
| | (AUC) |
| Treatment | Right paw |
| Compound 20 (10 mg/Kg) | 32 ± 8 |
| MTX (0.05 mg/Kg) | 33 ± 9 |
| Compound 20 (10 mg/Kg) + MTX | 53 ± 6 |
| (0.05 mg/Kg) | |

Results are expressed as the percentage of inhibition of the area under the curve (AUC) of the paw inflammation (measured by plethysmometry) in the period comprised between days 10 and 21 post-induction of arthritis. The percentage of inhibition for every group was calculated versus values from vehicle-treated rats. The table below depicts the mean and SEM of one experiment using 6 animals per group.

Results from Table 3 show that Compound 20 of the present invention inhibits the inflammation caused by experimental arthritis in rats. Furthermore, the co-administration of MTX and compound 20 resulted in an increased efficacy (65%) versus compound 20 alone, thus indicating the feasibility of administering the compound in patients treated with MTX.

1. A combination comprising (a) methotrexate and (b) a non-hepatotoxic DHODH inhibitor of formula (I): 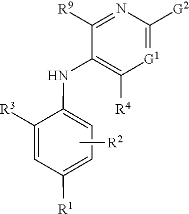
wherein:
R¹ is chosen from a hydrogen atom, halogen atoms, C₁₋₄ alkyl groups, C₃₋₄ cycloalkyl groups, —CF₃ groups, and —OCF₃ groups,
R² is chosen from a hydrogen atom, halogen atoms and C₁₋₄ alkyl groups,
R³ is chosen from —COOR⁵ groups, —CONHR⁵ groups, tetrazolyl groups, —SO₂NHR⁵ groups and —CONHSO₂R⁵ groups, wherein R⁵ is chosen from a hydrogen atom and linear and branched C₁₋₄ alkyl groups,
R⁴ is chosen from a hydrogen atom and C₁₋₄ alkyl groups,
R⁹ is chosen from a hydrogen atom and phenyl groups,
G¹ is chosen from N and CR⁶ wherein R⁶ is chosen from a hydrogen atom, halogen atoms, C₁₋₄ alkyl groups, C₃₋₄ cycloalkyl groups, C₁₋₄ alkoxy groups, —CF₃ groups, —OCF₃ groups, monocyclic N-containing C₅₋₇ heteroaryl groups, monocyclic N-containing C₃₋₇ heterocyclyl groups and C₆₋₁₀ aryl groups wherein the C₆₋₁₀ aryl groups are optionally substituted with one or more substituents chosen from halogen atoms and C₁₋₄ alkyl groups,
G² is chosen from:
a hydrogen atom, hydroxy groups, halogen atoms, C₃₋₄ cycloalkyl groups, C₁₋₄ alkoxy groups and —NR^{a}R^{b} groups, wherein
R^{a} is chosen from C₁₋₄ alkyl groups and R^{b} is chosen from C₁₋₄ alkyl groups and C₁₋₄ alkoxy-C₁₋₄ alkyl groups, or
R^{a} and R^{b} together with the nitrogen atom to which they are attached form a saturated 6 to 8 membered heterocyclic ring, wherein the 6 to 8 membered heterocyclic ring optionally contains one oxygen atom as an additional heteroatom,
monocyclic and bicyclic 5 to 10 membered heteroaromatic rings containing one or more nitrogen atoms, wherein the monocyclic and bicyclic 5 to 10 membered heteroaromatic rings are optionally substituted by one or more substituents chosen from halogen atoms, C₁₋₄ alkyl groups, C₁₋₄ alkoxy groups, C₃₋₄ cycloalkyl groups, C₃₋₄ cycloalkoxy groups, —CF₃ groups, —OCF₃ groups, and —CONR⁷R⁸ groups, wherein R⁷ and R⁸ are independently chosen from a hydrogen atom, linear and branched C₁₋₄ alkyl groups, C₃₋₇ cycloalkyl groups, or R⁷ and R⁸ together with the nitrogen atom to which they are attached form a group of formula 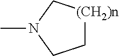
wherein n is an integer from 0 to 3,
and
phenyl groups which are optionally substituted by one or more substituents chosen from halogen atoms, C₁₋₄ alkyl groups, hydroxyl, C₁₋₄ alkoxy groups, C₃₋₄cycloalkyl groups, C₃₋₄ cycloalkoxy groups, cyano, —CF₃ groups, —OCF₃ groups, —CONR⁷R⁸ groups, oxadiazolyl groups, triazolyl groups, pyrazolyl groups and imidazolyl groups, wherein the oxadiazolyl groups, triazolyl groups, pyrazolyl groups and imidazolyl groups are optionally substituted by C₁₋₄ alkyl and C₃₋₇ cycloalkyl groups and wherein R⁷ and R⁸ are independently chosen from a hydrogen atom, linear and branched C₁₋₄ alkyl groups, C₃₋₇ cycloalkyl groups, or R⁷ and R⁸ together with the nitrogen atom to which they are attached form a group of formula 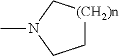
wherein n is an integer from 0 to 3
or, when G¹ represents CR⁶, G² together with R⁶ forms a non-aromatic C₅₋₁₀ carbocyclic group or a C₆₋₁₀ aryl group,
or a pharmaceutically acceptable salt or N-oxide thereof.

2. The combination according to claim 1, wherein R¹ is chosen from a hydrogen atom, fluorine atoms, chlorine atoms, bromine atoms, C₁₋₄ alkyl groups, C₃₋₄ cycloalkyl groups, and —CF₃ groups.

3. The combination according to claim 1, wherein R² is chosen from a hydrogen atom, halogen atoms and a methyl group.

4. The combination according to claim 1, wherein G¹ is chosen from nitrogen atoms, CCl, CF, CH, C(CH₃) groups, C(cyclopropyl) groups, C(phenyl) groups, and C(CF₃) groups.

5. The combination according to claim 1, wherein G² is chosen from:
a hydrogen atom, halogen atoms, C₃₋₄ cycloalkyl groups, C₁₋₂ alkoxy groups and —NR^{a}R^{b} groups, wherein
R^{a} is chosen from represents a C₁₋₂ alkyl groups and R^{b} is chosen from C₁₋₂ alkyl groups and C₁₋₂ alkoxy-C₁₋₂ alkyl groups, or R^{a} and R^{b} together with the nitrogen atom to which they are attached form a saturated 6 or 7 membered heterocyclic ring, wherein the saturated 6 or 7 membered heterocyclic ring optionally contains one oxygen atom as an additional heteroatom,
monocyclic and bicyclic 5 to 10 membered heteroaromatic rings containing one or more nitrogen atoms, wherein the monocyclic and bicyclic 5 to 10 membered heteroaromatic rings are optionally substituted by one or more substituents chosen from halogen atoms and C₁₋₄ alkyl groups,
and
phenyl groups which are optionally substituted by one, two or three substituents chosen from halogen atoms, C₁₋₄ alkyl groups, hydroxyl groups, C₁₋₄ alkoxy groups, C₃₋₄cycloalkyl groups, C₃₋₄ cycloalkoxy groups, cyano groups, —CF₃ groups, —OCF₃ groups, —CONR⁷R⁸ groups and oxadiazolyl groups, wherein the oxadiazolyl groups are optionally substituted by a C₁₋₄ alkyl groups and or C₃₋₇ cycloalkyl groups and wherein R⁷ and R⁸ are independently chosen from a hydrogen atom, linear and branched C₁₋₄ alkyl groups, C₃₋₄ cycloalkyl groups, or R⁷ and R⁸ together with the nitrogen atom to which they are attached form a group of formula 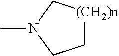
wherein n is 1 or 2,
or, when G¹ represents CR⁶, G² together with R⁶ forms a non-aromatic C₆ carbocyclic group or a phenyl group.

6. The combination according to claim 1, wherein G² is chosen from:
a hydrogen atom, a fluorine atom, cyclopropyl groups, methoxy groups, —NMeEt groups, —NEt₂ groups, —N(Me)—(CH₂)₂—O—CH₃ groups, 6-morpholinyl, azepan-1-yl groups, and piperidin-1-yl groups,
pyridinyl groups, pyrimidinyl groups, quinolinyl groups, and pyrazinyl rings optionally substituted with one or two substituents chosen from Me and F,
and
phenyl groups optionally substituted by one, two or three substituents chosen from fluorine groups, chlorine groups, methyl groups, hydroxyl groups, methoxy groups, ethoxy groups, isopropyloxy groups, cyclopropyl groups, cyclopropyloxy groups, cyano groups, —CF₃ groups, —OCF₃ groups, oxadiazolyl groups, and —CONR⁷R⁸ groups, wherein the oxadiazolyl groups are optionally substituted by a methyl group and wherein R⁷ and R⁸ are independently chosen from a hydrogen atom, methyl groups, isopropyl groups, cyclopropyl groups, or R⁷ and R⁸ together with the nitrogen atom to which they are attached form a group of formula 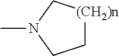
wherein n is 1,
or, when G¹ represents CR⁶, G² together with R⁶ forms a non-aromatic C₆ carbocyclic group or a phenyl group.

7. The combination according to claim 1, wherein G² is chosen from methoxy groups, cyclopropyl groups, and optionally substituted phenyl groups, pyridyl groups, quinolynyl groups, pyrimidinyl groups, and pyrazinyl groups.

8. The combination according to claim 1, wherein:
R⁹ represents a hydrogen atom, and
G² is chosen from:
monocyclic and bicyclic 5 to 10 membered heteroaromatic rings containing a nitrogen atom, wherein the monocyclic and bicyclic 5 to 10 membered heteroaromatic rings are optionally substituted by one or more substituents chosen from halogen atoms, C₁₋₄ alkyl groups, C₁₋₄ alkoxy groups, C₃₋₄ cycloalkyl groups, C₃₋₄ cycloalkoxy groups, —CF₃ groups, —OCF₃ groups, and —CONR⁷R⁸ groups, wherein R⁷ and R⁸ are independently chosen from a hydrogen atom, linear and branched C₁₋₄ alkyl groups, C₃₋₇ cycloalkyl groups, or R⁷ and R⁸ together with the nitrogen atom to which they are attached form a group of formula 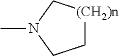
wherein n is an integer from 0 to 3,
and
phenyl groups optionally substituted by one or more substituents chosen from halogen atoms, C₁₋₄ alkyl groups, C₁₋₄ alkoxy groups, C₃₋₄cycloalkyl groups, C₃₋₄ cycloalkoxy groups, —CF₃ groups, —OCF₃ groups, —CONR⁷R⁸ groups, oxadiazolyl groups, triazolyl groups, pyrazolyl groups, and imidazolyl groups, wherein the oxadiazolyl groups, triazolyl groups, pyrazolyl groups, and imidazolyl groups are optionally substituted by C₁₋₄ alkyl groups and C₃₋₇ cycloalkyl groups and wherein R⁷ and R⁸ are independently chosen from hydrogen atoms, linear and branched C₁₋₄ alkyl groups, C₃₋₇ cycloalkyl groups, or R⁷ and R⁸ together with the nitrogen atom to which they are attached form a group of formula 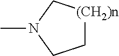
wherein n is an integer from 0 to 3.

9. The combination according to claim 1, wherein R¹ is chosen from C₁₋₄ alkyl groups, C₃₋₄ cycloalkyl groups, and —CF₃ groups.

10. The combination according to claim 1, wherein R² is chosen from a hydrogen and halogen atoms.

11. The combination according to claim 1, wherein R³ is chosen from COOR⁵ groups, —CONHR⁵ groups, and tetrazolyl groups.

12. The combination according to claim 1, wherein R⁴ is chosen from a hydrogen atom and methyl groups.

13. The combination according to claim 1, wherein R⁹ represents a hydrogen atom.

14. The combination according to claim 1, wherein G¹ is chosen from nitrogen atoms and CH, C(CH₃) groups, C(cyclopropyl) groups, C(phenyl) groups, and C(CF₃) groups.

15. The combination according to claim 1, wherein G² is chosen from optionally substituted phenyl groups, pyridyl groups, quinolynyl groups, pyrimidinyl groups, and pyrazinyl groups.

16. The combination according to claim 1, wherein R¹ is chosen from methyl groups and cyclopropyl groups, R² represents a hydrogen atom, R³ is a COOH group, R⁴ chosen from a hydrogen atom and methyl groups, G¹ is chosen from nitrogen atoms and CH, C(CH₃) groups, C(cyclopropyl) groups, C(phenyl) groups, and C(CF₃) groups and G² is chosen from optionally substituted phenyl groups, 4-pyridyl groups, 5-quinolynyl groups, and 2-pyrazinyl groups.

17. The combination according to claim 16, wherein R⁹ represents a hydrogen atom.

18. The combination according to claim 1, wherein R¹ is chosen from methyl groups cyclopropyl groups, R² represents a hydrogen atom, R³ is a COOH group, R⁴ represents a hydrogen atom, G¹ is chosen from nitrogen atoms and CH, C(CH₃) groups and C(CF₃) groups, and G² is chosen from phenyl groups grew optionally substituted with one or two substituents chosen from chloro, fluoro, methoxy, ethoxy, isopropoxy, trifluoromethoxy and —CONR⁷R⁸, wherein R⁷ is hydrogen and R⁸ is cyclopropyl or R⁷ and R⁸ together with the nitrogen atom to which they are attached form a group of formula 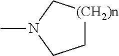
wherein n is 1.

19. The combination according to claim 1, wherein the DHODH inhibitor is chosen from:
5-cyclopropyl-2-(2-phenylpyrimidin-5-ylamino)benzoic acid;
2-(6-Cyclopropyl-5-methylpyridin-3-ylamino)-5-methylbenzoic acid;
5-(2-Carboxy-4-cyclopropylphenylamino)-3-methyl-2-phenylpyridine 1-oxide;
5-Methyl-2-(6-(3-(trifluoromethyl)phenyl)pyridin-3-ylamino)benzoic acid;
5-cyclopropyl-2-(6-hydroxy-5-phenylpyridin-3-ylamino)benzoic acid;
5-cyclopropyl-2-(2-(2,6-difluoro-4-hydroxyphenyppyrimidin-5-ylamino)benzoic acid;
5-Cyclopropyl-2-(6-methoxy-5-phenylpyridin-3-ylamino)benzoic acid;
2-(5-Fluoro-6-phenylpyridin-3-ylamino)-5-methylbenzoic acid;
2-(6-(Ethyl(methyl)amino)-5-methylpyridin-3-ylamino)-5-methylbenzoic acid;
5-Cyclopropyl-2-(3′-fluoro-2,4′-bipyridin-5-ylamino)benzoic acid;
2-(6-(Diethylamino)-5-methylpyridin-3-ylamino)-5-methylbenzoic acid;
2-(6-((2-Methoxyethyl)(methyl)amino)-5-methylpyridin-3-ylamino)-5-methylbenzoic acid;
2-(5-Chloro-6-phenylpyridin-3-ylamino)-5-methylbenzoic acid;
5-Cyclopropyl-2-(2-(2-cyclopropylphenyl)pyrimidin-5-ylamino)benzoic acid;
5-cyclopropyl-2-(5-phenylpyridin-3-ylamino)benzoic acid;
5-methyl-2-(quinolin-3-ylamino)benzoic acid;
5-methyl-2-(5,6,7,8-tetrahydroquinolin-3-ylamino)benzoic acid;
2-(5-Chloro-2-phenylpyridin-3-ylamino)-5-methylbenzoic acid;
5-Cyclopropyl-2-(5,6-diphenylpyridin-3-ylamino)benzoic acid;
5-cyclopropyl-2-(2-(2,6-difluorophenyl)pyrimidin-5-ylamino)benzoic acid;
5-Cyclopropyl-2-(5-methylpyridin-3-ylamino)benzoic acid;
2-(2-(3-Cyclopropoxyphenyl)pyrimidin-5-ylamino)-5-cyclopropylbenzoic acid;
5-Methyl-2-(6-morpholinopyridin-3-ylamino)benzoic acid;
5-Methyl-2-(5-methyl-6-morpholinopyridin-3-ylamino)benzoic acid;
5-cyclopropyl-2-(6-cyclopropyl-5-phenylpyridin-3-ylamino)benzoic acid;
2-(6-(2-Cyclopropylphenyl)-5-methylpyridin-3-ylamino)-5-methylbenzoic acid;
2-(6-(2-Cyanophenyl)-5-methylpyridin-3-ylamino)-5-methylbenzoic acid;
2-(2-(3-Chlorophenyl)pyrimidin-5-ylamino)-5-cyclopropylbenzoic acid;
5-Methyl-2-(6-phenyl-5-(trifluoromethyl)pyridin-3-ylamino)benzoic acid;
5-Methyl-2-(5-methyl-6-(piperidin-1-yl)pyridin-3-ylamino)benzoic acid;
2-(6-(Azepan-1-yl)-5-methylpyridin-3-ylamino)-5-methylbenzoic acid;
2-(6-(3-Methoxyphenyl)-5-phenylpyridin-3-ylamino)-5-methylbenzoic acid;
2-(2,3′-bipyridin-5-ylamino)-5-cyclopropylbenzoic acid;
2-(3′-chloro-2,4′-bipyridin-5-ylamino)-5-methylbenzoic acid;
5-Methyl-2-(3-methyl-2,2′-bipyridin-5-ylamino)benzoic acid;
2-(5,6-Difluoropyridin-3-ylamino)-5-methylbenzoic acid;
2-(6-(3-Methoxphenyl)pyridin-3-ylamino)benzoic acid;
2-(6-(3-Ethoxyphenyl)pyridin-3-ylamino)benzoic acid;
2-(6-(3-Ethoxyphenyl)pyridin-3-ylamino)-5-fluorobenzoic acid;
2-(6-(3-Ethoxyphenyl)-5-methylpyridin-3-ylamino)benzoic acid;
2-(6-(3-Ethoxyphenyl)pyridin-3-ylamino)-5-methylbenzoic acid;
2-(6-(3-Ethoxyphenyl)-5-methylpyridin-3-ylamino)-5-methylbenzoic acid;
2-(6-(3-Ethoxy-2-fluorophenyl)pyridin-3-ylamino)benzoic acid;
2-(6-(3-Ethoxyphenyl)-4-methylpyridin-3-ylamino)-5-methylbenzoic acid;
2-(6-(3-Ethoxyphenyl)-4-methylpyridin-3-ylamino)benzoic acid;
5-Bromo-2-(6-(3-ethoxyphenyl)pyridin-3-ylamino)benzoic acid;
5-Chloro-2-(6-(3-ethoxyphenyl)pyridin-3-ylamino)benzoic acid;
2-(6-(5-Ethoxy-2-fluorophenyl)pyridin-3-ylamino)benzoic acid;
2-(6-(3-Ethoxyphenyl)-5-methylpyridin-3-ylamino)-5-(trifluoromethyl)benzoic acid;
2-(6-(3-Methoxyphenyl)-5-methylpyridin-3-ylamino)-5-(trifluoromethyl)benzoic acid;
2-(6-(3-Methoxyphenyl)-5-methylpyridin-3-ylamino)-5-methylbenzoic acid;
2-(6-(3-Methoxyphenyl)-5-methylpyridin-3-ylamino)-6-methylbenzoic acid;
5-Fluoro-2-(6-(3-methoxyphenyl)-5-methylpyridin-3-ylamino)benzoic acid;
2-(6-(5-Ethoxy-2-fluorophenyl)pyridin-3-ylamino)-5-methylbenzoic acid;
2-(6-(2-Fluoro-5-methoxyphenyl)-5-methylpyridin-3-ylamino)-5-methylbenzoic acid;
Ethyl 2-(6-(2-fluoro-5-methoxyphenyl)-5-methylpyridin-3-ylamino)-5-methylbenzoate;
2-(6-(2-Fluorophenyl)pyridin-3-ylamino)-5-methylbenzoic acid;
2-(6-(3-Methoxyphenyl)-5-phenylpyridin-3-ylamino)-5-methylbenzoic acid;
Ethyl 2-(6-(3-methoxyphenyl)-5-phenylpyridin-3-ylamino)-5-methylbenzoate;
5-Methyl-2-(5-methyl-6-phenylpyridin-3-ylamino)benzoic acid;
Ethyl 5-methyl-2-(5-methyl-6-phenylpyridin-3-ylamino)benzoate;
5-Methyl-2-(5-methyl-6-(3-(trifluoromethoxy)phenyl)pyridin-3-ylamino)benzoic acid;
Ethyl 5-methyl-2-(5-methyl-6-(3-(trifluoromethoxy)phenyl)pyridin-3-ylamino)benzoate;
2-(5-Cyclopropyl-6-(3-methoxyphenyl)pyridin-3-ylamino)-5-methylbenzoic acid;
Ethyl 2-(5-cyclopropyl-6-(3-methoxyphenyl)pyridin-3-ylamino)-5-methylbenzoate;
2-(6-(2-Fluoro-5-isopropoxyphenyl)pyridin-3-ylamino)-5-methylbenzoic acid;
2-(6-(3-Isopropoxyphenyl)-5-methylpyridin-3-ylamino)-5-methylbenzoic acid;
Ethyl 2-(6-(3-isopropoxyphenyl)-5-methylpyridin-3-ylamino)-5-methylbenzoate;
2-(6-(3-Cyclopropoxyphenyl)-5-methylpyridin-3-ylamino)-5-methylbenzoic acid;
tent-Butyl 2-(6-(3-cyclopropoxyphenyl)-5-methylpyridin-3-ylamino)-5-methylbenzoate;
2-(6-(2-Chlorophenyl)-5-methylpyridin-3-ylamino)-5-methylbenzoic acid;
tert-Butyl 2-(6-(2-chlorophenyl)-5-methylpyridin-3-ylamino)-5-methylbenzoate;
2-(6-(3-Carbamoylphenyl)-5-methylpyridin-3-ylamino)-5-methylbenzoic acid;
Ethyl 2-(6-(3-carbamoylphenyl)-5-methylpyridin-3-ylamino)-5-methylbenzoate;
2-(6-(2-Fluoro-5-methoxyphenyl)-4-methylpyridin-3-ylamino)-5-methylbenzoic acid;
Ethyl 2-(6-(2-fluoro-5-methoxyphenyl)-4-methylpyridin-3-ylamino)-5-methylbenzoate;
2-(6-(3-Methoxyphenyl)-5-(trifluoromethyl)pyridin-3-ylamino)-5-methylbenzoic acid;
Ethyl 2-(6-(3-methoxyphenyl)-5-(trifluoromethyl)pyridin-3-ylamino)-5-methylbenzoate;
2-(6-(3-(Dimethylcarbamoyl)phenyl)-5-methylpyridin-3-ylamino)-5-methylbenzoic acid;
Ethyl 2-(6-(3-(dimethylcarbamoyl)phenyl)-5-methylpyridin-3-ylamino)-5-methylbenzoate;
2-(6-(3-Isopropoxyphenyl)-5-methylpyridin-3-ylamino)-3-methylbenzoic acid;
tert-Butyl 2-(6-(3-isopropoxyphenyl)-5-methylpyridin-3-ylamino)-3-methylbenzoate;
3-Methyl-2-(5-methyl-6-phenylpyridin-3-ylamino)benzoic acid;
tert-Butyl 3-methyl-2-(5-methyl-6-phenylpyridin-3-ylamino)benzoate;
2-(6-(2-Chlorophenyl)pyridin-3-ylamino)-5-methylbenzoic acid;
tert-Butyl 2-(6-(2-chlorophenyl)pyridin-3-ylamino)-5-methylbenzoate;
3-Fluoro-2-(6-(3-methoxyphenyl)-5-methylpyridin-3-ylamino)benzoic acid;
tert-Butyl 3-fluoro-2-(6-(3-methoxyphenyl)-5-methylpyridin-3-ylamino)benzoate;
5-Cyclopropyl-2-(5-methyl-6-(3-(trifluoromethoxy)phenyl)pyridin-3-ylamino)benzoic acid;
Ethyl 5-cyclopropyl-2-(5-methyl-6-(3-(trifluoromethoxy)phenyl)pyridin-3-ylamino)benzoate;
5-Cyclopropyl-2-(5-methyl-6-phenylpyridin-3-ylamino)benzoic acid;
Ethyl 5-cyclopropyl-2-(5-methyl-6-phenylpyridin-3-ylamino)benzoate;
5-Methyl-2-(5-methyl-6-(2-(trifluoromethyl)phenyl)pyridin-3-ylamino)benzoic acid;
tert-Butyl 5-methyl-2-(5-methyl-6-(2-(trifluoromethyl)phenyl)pyridin-3-ylamino)benzoate;
2-(6-(3-Chlorophenyl)-5-methylpyridin-3-ylamino)-5-methylbenzoic acid;
tert-Butyl 2-(6-(3-chlorophenyl)-5-methylpyridin-3-ylamino)-5-methylbenzoate;
2-(6-(2-Fluorophenyl)-5-methylpyridin-3-ylamino)-5-methylbenzoic acid;
tert-Butyl 2-(6-(2-fluorophenyl)-5-methylpyridin-3-ylamino)-5-methylbenzoate;
5-Methyl-2-(5-methyl-6-(quinolin-5-yl)pyridin-3-ylamino)benzoic acid;
tert-Butyl 5-methyl-2-(5-methyl-6-(quinolin-5-yl)pyridin-3-ylamino)benzoate;
2-(3′-Fluoro-3-methyl-2,4′-bipyridin-5-ylamino)-5-methylbenzoic acid;
tert-Butyl 2-(3′-fluoro-3-methyl-2,4′-bipyridin-5-ylamino)-5-methylbenzoate;
5-Methyl-2-(5-methyl-6-(pyrazin-2-yl)pyridin-3-ylamino)benzoic acid;
tert-Butyl 5-methyl-2-(5-methyl-6-(pyrazin-2-yl)pyridin-3-ylamino)benzoate;
5-Cyclopropyl-2-(6-phenyl-5-(trifluoromethyl)pyridin-3-ylamino)benzoic acid;
Ethyl 5-cyclopropyl-2-(6-phenyl-5-(trifluoromethyl)pyridin-3-ylamino)benzoate;
5-Cyclopropyl-2-(6-(3-methoxyphenyl)-5-(trifluoromethyl)pyridin-3-ylamino)benzoic acid;
Ethyl 5-cyclopropyl-2-(6-(3-methoxyphenyl)-5-(trifluoromethyl)pyridin-3-ylamino)benzoate;
5-Chloro-2-(6-(2-fluorophenyl)pyridin-3-ylamino)benzoic acid;
5-Chloro-2-(6-(2-chlorophenyl)pyridin-3-ylamino)benzoic acid;
5-Chloro-2-(6-(quinolin-5-yl)pyridin-3-ylamino)benzoic acid;
2-(6-(2-Chlorophenyl)pyridin-3-ylamino)-5-cyclopropylbenzoic acid;
Ethyl 2-(6-(2-chlorophenyl)pyridin-3-ylamino)-5-cyclopropylbenzoate;
5-Chloro-2-(6-(2-(trifluoromethyl)phenyl)pyridin-3-ylamino)benzoic acid;
5-Fluoro-2-(6-(2-(trifluoromethyl)phenyl)pyridin-3-ylamino)benzoic acid;
2-(3′-Fluoro-2,4′-bipyridin-5-ylamino)-5-methylbenzoic acid;
2-(2-(2-Fluorophenyl)pyrimidin-5-ylamino)-5-methylbenzoic acid;
tert-Butyl 2-(2-(2-fluorophenyl)pyrimidin-5-ylamino)-5-methylbenzoate;
2-(6-(2,6-Difluorophenyl)pyridin-3-ylamino)-5-methylbenzoic acid;
Ethyl 2-(6-(2,6-difluorophenyl)pyridin-3-ylamino)-5-methylbenzoate;
2-(2-(2-Chlorophenyl)pyrimidin-5-ylamino)-5-cyclopropylbenzoic acid;
Methyl 2-(2-(2-chlorophenyl)pyrimidin-5-ylamino)-5-cyclopropylbenzoate;
2-(2-(2-Chlorophenyl)pyrimidin-5-ylamino)-5-methylbenzoic acid;
tert-Butyl 2-(2-(2-chlorophenyl)pyrimidin-5-ylamino)-5-methylbenzoate;
5-Methyl-2-(5-methyl-6-(3-(pyrrolidine-1-carbonyl)phenyl)pyridin-3-ylamino)benzoic acid;
2-(6-(3-(Cyclopropylcarbamoyl)phenyl)-5-methylpyridin-3-ylamino)-5-methylbenzoic acid;
5-Cyclopropyl-2-(2-(2-fluorophenyl)pyrimidin-5-ylamino)benzoic acid;
2-(2-(2-trifluoromethylphenyl)pyrimidin-5-ylamino)-5-cyclopropylbenzoic acid;
2-(2-o-tolylpyrimidin-5-ylamino)-5-cyclopropylbenzoic acid;
2-(2-(2-cyclopropoxyphenyl)pyrimidin-5-ylamino)-5-cyclopropylbenzoic acid;
2-(2-(2,5-difluorophenyl)pyrimidin-5-ylamino)-5-cyclopropylbenzoic acid;
2-(2-(2,3-difluorophenyl)pyrimidin-5-ylamino)-5-cyclopropylbenzoic acid;
2-(2-(2-fluoro-5-chlorophenyl)pyrimidin-5-ylamino)-5-cyclopropylbenzoic acid;
2-(2-(2-trifluoromethylphenyl)pyrimidin-5-ylamino)-5-methylbenzoic acid;
2-(2-(2-fluoro-5-trifluoromethoxyphenyl)pyrimidin-5-ylamino)-5-cyclopropylbenzoic acid;
2-(6-(2-trifluoromethylphenyl)pyridin-3-ylamino)-5-methylbenzoic acid;
2-(6-phenylpyridin-3-ylamino)-5-cyclopropylbenzoic acid;
2-(6-(2-fluorophenyl)pyridin-3-ylamino)-5-cyclopropylbenzoic acid;
2-(6-(3,5-difluoropyridin-4-yl)pyridin-3-ylamino)-5-methylbenzoic acid;
2-(6-(3-cyclopropylcarbamoylphenyl)pyridin-3-ylamino)-5-methylbenzoic acid;
2-(6-(2,4-difluorophenyl)pyridin-3-ylamino)-5-methylbenzoic acid;
2-(6-(2,5-difluorophenyl)pyridin-3-ylamino)-5-methylbenzoic acid;
2-(6-(2-fluorophenyl)pyridin-3-ylamino)-5-cyclopropyl-3-fluorobenzoic acid;
2-(6-(2,3,6-trifluorophenyl)pyridin-3-ylamino)-5-methylbenzoic acid;
2-(6-(3-(5-methyl-1,3,4-oxadiazol-2-yOphenyl)pyridin-3-ylamino)-5-methylbenzoic acid;
2-(5-methyl-6-(pyrimidin-5-yl)pyridin-3-ylamino)-5-methylbenzoic acid;
2-(6-(2,3-difluorophenyl)pyridin-3-ylamino)-5-methylbenzoic acid;
2-(6-(5-fluoro-2-methoxyphenyl)-5-methylpyridin-3-ylamino)-5-methylbenzoic acid;
2-(6-(4-carbamoylphenyl)-5-methylpyridin-3-ylamino)-5-methylbenzoic acid,
or a pharmaceutically acceptable salt or N-oxide thereof.

20. The combination according to claim 1, wherein the DHODH inhibitor is 5-Methyl-2-(6-(3-(trifluoromethyl)phenyl)pyridin-3-ylamino)benzoic acid or a pharmaceutically acceptable salt or N-oxide thereof.

21. The combination according to claim 1, wherein the DHODH inhibitor is 5-cyclopropyl-2-(2-(2,6-difluorophenyl)pyrimidin-5-ylamino)benzoic acid or a pharmaceutically acceptable salt or N-oxide thereof.

22. The combination according to claim 1, wherein the DHODH inhibitor is 2-(6-(2,6-difluorophenyl)pyridin-3-ylamino)-5-methylbenzoic acid or a pharmaceutically acceptable salt or N-oxide thereof.

23. The combination according to claim 1, wherein the DHODH inhibitor is 2-(6-(3-(cyclopropylcarbamoyl)phenyl)-5-methylpyridin-3-ylamino)-5-methylbenzoic acid or a pharmaceutically acceptable salt or N-oxide thereof.

24. The combination according to claim 1, wherein the active ingredients (a) methotrexate, and (b) non-hepatotoxic inhibitor, form part of a single pharmaceutical composition.

25. The combination according to claim 1, further comprising at least one compound (c) chosen from:
(i) Anti-TNF-alpha monoclonal antibodies;
(ii) TNF-alpha Antagonists;
(iii) Calcineurin (PP-2B) Inhibitors/INS Expression Inhibitors;
(iv) IL-1 Receptor Antagonists;
(v) Anti-CD20 monoclonal antibodies;
(vi) p38 Inhibitors;
(vii) NF-kappaB (NFKB) Activation Inhibitors;
(viii) dihydrofolate reductase (DHFR) inhibitors;
(ix) Janus kinase (JAK) inhibitors;
(x) MEK inhibitors; and
(xi) Sphingosine-1 phosphate receptor agonists;
(xii) Interferons comprising Interferon beta 1a, and interferons comprising Interferon beta 1b;
(xiii) Immunomodulators; and
(xiv) Adenosine aminohydrolase inhibitors.

26-27. (canceled)

28. A product comprising (a) methotrexate and (b) a non-hepatotoxic DHODH inhibitor according to claim 1.

29. The product according to claim 28, further comprising at least one compound (c) chosen from:
(i) Anti-TNF-alpha monoclonal antibodies;
(ii) TNF-alpha Antagonists;
(iii) Calcineurin (PP-2B) Inhibitors/INS Expression;
(iv) IL-1 Receptor Antagonists;
(vi) p38 Inhibitors;
(vii) NF-kappaB (NFκB) Activation Inhibitors;
(viii) dihydrofolate reductase (DHFR) inhibitors;
(ix) Janus kinase (JAK) inhibitors;
(x) MEK inhibitors; and
(xi) Sphingosine-1 phosphate receptor agonists;
(xii) Interferons comprising Interferon beta 1a, and interferons comprising Interferon beta 1b;
(xiii) Immunomodulators; and
(xiv) Adenosine aminohydrolase inhibitors.

30. A kit of parts comprising (a) methotrexate, and (b) a non-hepatotoxic DHODH inhibitor according to claim 1, together with instructions for simultaneous, separate or sequential administration of methotrexate and the non-hepatotoxic DHODH inhibitor.

31. The kit according to claim 30, further comprising at least one compound (c) chosen from:
(i) Anti-TNF-alpha monoclonal antibodies;
(ii) TNF-alpha Antagonists;
(iii) Calcineurin (PP-2B) Inhibitors/INS Expression;
(iv) IL-1 Receptor Antagonists;
(vi) p38 Inhibitors;
(vii) NF-kappaB (NFKB) Activation Inhibitors;
(viii) dihydrofolate reductase (DHFR) inhibitors;
(ix) Janus kinase (JAK) inhibitors;
(x) MEK inhibitors; and
(xi) Sphingosine-1 phosphate receptor agonists;
(xii) Interferons comprising Interferon beta 1a, and interferons comprising Interferon beta 1b;
(xiii) Immunomodulators; and
(xiv) Adenosine aminohydrolase inhibitors.

32. A package comprising the product of claim 28.

33. The package according to claim 32, which further comprising at least one compound (c) chosen from:
(i) Anti-TNF-alpha monoclonal antibodies;
(ii) TNF-alpha Antagonists;
(iii) Calcineurin (PP-2B) Inhibitors/INS Expression;
(iv) IL-1 Receptor Antagonists;
(vi) p38 Inhibitors;
(vii) NF-kappaB (NFKB) Activation Inhibitors;
(viii) dihydrofolate reductase (DHFR) inhibitors;
(ix) Janus kinase (JAK) inhibitors;
(x) MEK inhibitors; and
(xi) Sphingosine-1 phosphate receptor agonists;
(xii) Interferons comprising Interferon beta 1a, and interferons comprising Interferon beta 1b;
(xiii) Immunomodulators; and
(xiv) Adenosine aminohydrolase inhibitors.

34. A method for preparing a medicament comprising: combining (b) a non-hepatotoxic DHODH inhibitor according to claim 1, with (a) methotrexate.

35. (canceled)

36. The method according to claim 41, wherein the methotrexate (a) is administered at a dose of 0.015 to 3 mg/kg/week and the non-hepatotoxic DHODH inhibitor (b) is administered at a dose of 0.03 to 30 mg/kg/day.

37. (canceled)

38. The method according to claim 41, wherein the condition that would be aggravated by hepatotoxicity is chosen from liver fibrosis, hepatitis, cirrhosis and liver cancer.

39. (canceled)

40. A method of treating a human or animal patient suffering from or susceptible to a pathological condition or disease susceptible to amelioration by inhibition of dehydroorotate dehydrogenase, wherein the method comprises simultaneously, separately or sequentially administering to the human or animal patient in need thereof, a therapeutically effective amount of (a) methotrexate and (b) a non-heptoxic DHODH inhibitor according to claim 1.

41. The method according to claim 40, wherein the human or animal patient is suffering from or susceptible to hepatic impairment or a condition that would be aggravated by hepatotoxicity.

42-43. (canceled)

44. The method according to claim 40, wherein the pathological condition or disease is chosen from rheumatoid arthritis, psoriatic arthritis, ankylosing spondilytis, multiple sclerosis, Wegener's granulomatosis, systemic lupus erythematosus, psoriasis and sarcoidosis.
